(19) United States
(12) Patent Application Publication
(10) Pub. No.: US 2020/0398077 A1
(43) Pub. Date: Dec. 24, 2020

(54) SYSTEM AND METHOD TO ERADICATE A MAJORITY OF DISEASE-CAUSING GERMS AND PRODUCE AVIRULENT PHENOTYPE SURVIVORS SUSCEPTIBLE TO MICROBICIDES USING A LIGHT DOSE REGIMENT

(71) Applicant: GAMA Therapeutics LLC, Worcester, MA (US)

(72) Inventor: Anthony Ball, Mendon, MA (US)

(21) Appl. No.: 16/906,143

(22) Filed: Jun. 19, 2020

Related U.S. Application Data
(60) Provisional application No. 62/864,762, filed on Jun. 21, 2019.

(51) Int. Cl. A61N 5/06; A61L 2/26; A61L 2/00

(52) U.S. Cl. CPC A61N 5/0624; A61N 5/0616; A61L 2202/11; A61L 2/0047; A61N 2005/0661; A61L 2/26

(57) ABSTRACT

A system to eradicate a majority of disease-causing germs and produce avirulent phenotype survivors susceptible to microbicides using a light dose regiment. The system includes one or more processors and one or more light sources coupled to the one or more processors, The one or more processors is configured to control the one or more light sources to apply the light dose regiment including a) continuously applying a pre-treatment of non-lethal UVA light to the disease-causing germs to increase the production of pigment to the disease-causing germs, b) simultaneously applying pulsed UVA light and continuous UVC light to the disease-causing germs having the increased pigment therein to eradicate the majority of the disease-causing germs; and c) cycling steps a) and b) a selected number of times to generate avirulent phenotype survivors susceptible to microbicides.

### RELATED APPLICATIONS

This application claims benefit of and priority to U.S. Provisional Application Ser. No. 62/864,762 filed Jun. 21, 2019 under 35 U.S.C. §§ 119, 120, 363, 365, and 37 C.F.R. § 1.55 and § 1.78, which is incorporated herein by this reference.

### GOVERNMENT RIGHTS

This invention was made with U.S. Government support under Contract No. W81XWH-18-C-0003 awarded by the U.S. Army Medical Research Acquisition Activity (USAMRAA). The Government has certain rights in the subject invention.

### FIELD OF THE INVENTION

This invention relates to a system and method to eradicate a majority of disease causing germs and produce avirulent phenotype survivors susceptible to microbicides using a light dose regiment.

### BACKGROUND OF THE INVENTION

As disclosed herein, microbicides include all antibiotics and agents such as antifungals, e.g., azoles, which inhibit the synthesis of ergosterol, polyenes, which interact with fungal membrane sterols physicochemically, and 5-fluorocytosine, which inhibits macromolecular synthesis, and the like. Microbicides include antibiotics which have specific mechanisms of action and also include antiseptics which generalized do not have specific targets. For example, flouroquinoline and beta-lactams are antibiotics, and target key bacterial pathways and have no effect on fungi or viruses, typically about antibiotic drug resistance refers to the common classes of antibiotics, whereas antiseptics are broadly acting. Classes of antibiotics include: Aminoglycosides, Carbapenems, Cephalosporins, Fluoroquinolones, Glycopeptides and Lipoglycopeptides (such as vancomycin), Ketolides (such as telithromycin), Macrolides (such as Erythromycin), Monobactams (Aztreonam), Oxazolidinones (such as Linezolid and Tedizolid), Penicillins, Polypeptides, Rifamycins, Sulfonamides, Streptogramins (such as Quinupristin and Dalfopristin), Tetracyclines, Carhapenems, Cephalosporins, Mortobactams. Penicillins are subclasses of beta-lactam antibiotics, a class of antibiotics characterized by a chemical structure called a beta-lactam ring, Chloramphenicol, Clindamycin, Daptomycin, Fosfomycin, Metronidazole, Nitrofurantoin, and Tigecycline are other antibiotics that do not fit into the classes listed above.

Microbicides also include non-specific agents such as antiseptics, e.g., alcohols, quaternary ammonium compounds, chlorhexidine and other diguanides, antibacterial dyes, chlorine and hypochlorites, inorganic iodine compounds, metals, peroxides and permanganates, halogenated phenol derivatives and quinolone derivatives, and the like.

Microbicides are used to prevent and treat infections caused by disease-causing germs. As disclosed herein, disease-causing germs include disease-causing or pathogenic bacteria and fungi. As is well known, disease-causing germs can mutate and become resistant to microbicides. Resistance of disease-causing germs to microbicides is rising to dangerous levels worldwide. Disease-causing germs are continuously developing new resistance mechanisms to microbicides. The result may include ineffective medical treatment, increased time spent in hospitals, increased medical costs, and increased mortality.

Each year drug-resistant bacteria are responsible for the infection of about 25,000 people in Europe, about 23,000 in the U.S. and about 700,000 globally. See, e.g., Tackling Drug-Resistant Infections Globally: Final Report and Recommendations, The Review on Antimicrobial Resistance chaired by O'neill, J., London, NWI 2BE (2016), available from: https://amr-review.org./sites/default/files/160525_Final%20paper_with%20cover.pdf, incorporated by reference herein. Methicillin-resistant Staphylococcus (S. ateretis) (MRSA), extended spectrum beta-lactamases (ESEL) producing Enterobacteriaceae and P. aeruginosa are key pathogens implicated in difficult-to-treat infections. Overall, infection diagnosis and treatment is typically based on the presence of clinical findings and treatment typically requires management from highly skilled interdisciplinary experts including infectious disease specialists, surgeons, nurses, and the like, as well as time-consuming clinic visits and extensive homecare. A multitude of antimicrobial resistance (AMR) mechanisms dictate the lethality of bacterial infections. Antibiotic failure is driven by virulence factors involving cell surface components and/or secreted determinants as well as the adaptability to antimicrobials. See, e.g., Bernardini et al., The Intrinsic Resistoine of Bacterial Pathogens, Frontiers In Microbiology, 4:103; doi: 10.3389/fmicb.2013.00103. Pub ed PMID: 23641241(2013), incorporated by reference herein.

A biofilm is an expolysaccharide matrix containing microbial cells such as bacteria, fungi, or both, and can be multi-species. The biofilm has long been attributed to increased resistance, being difficult to eradicate with antibiotics and the source of many difficult to address and reoccurring infections, often referred to as recalcitrance. The classic hallmark of antibiotic resistance is the microbe's ability to grow and replicate in the presence of increased concentrations of an antimicrobial agent. However, as posited by Kim Lewis, the argument can be made that, while a biofilm may be comprised of microorganisms possessing drug resistance genes the biofilm itself does not confer drug “resistance” per say because the biofilm itself does not confer antibiotic resistance. Instead, the biofilm confers factors that reduce the effectiveness of the antibiotics but not a growth advantage in the presence of the antibiotic. Factors include restricted penetration of the antimicrobial due to the expolysaccharide matrix that blocks diffusion of large molecules, antimicrobial protein lysozyme, and blood complement. Smaller agents, such as defensins, are also blocked and positively charged agents, such as aminoglycosides, are also blocked by the negative charge of the matrix. However this only postpones cell death and does not confer long-term protection and other agents such as fluoroquinolones readily penetrate the biofilm. Combined with inactivation, such as catalase production to neutralize hydrogen peroxide, a multiplicative protection barrier is formed by the biofilm. This explains why biofilm populations of Pseudomonas aeruginosa are more protected against peroxides even though catalase production is lower in P. aeruginosa biofilms. Any resistance mechanism involving modification of the agent, such as acetylation of aminoglycosides, will be more effective inside the diffusion barrier of a biofilm. Finally, another multiply factor owing to increased tolerance of biofilms to microbicides agents is the expression of membrane translocases, the so-called multidrug resistance pumps. AcrAB forms a translocase an efflux pump is a transmembrane protein. The AcrAB-TolC MDR pump in Escherchia coli for example acts in concert with the chloramphenicol efflux pump (CmlA) to export chloramphenicol from the cell and this combination of drug efflux with the diffusion barrier works to collectively confer an advantage over free-floating planktonic populations. Universally, almost all microbicides are more effective at killing rapidly growing cells, and in some cases, it is required, such as with penicillin which does not kill non-growing cells. Exceptions include beta lactams, cephalosporins, aminoglycosides, and fluoroquinolones but slow growth still contributes to biofilm resistance to these agents. Taken these concepts into account Kim Lewis proposed a sub-population of cells, present within the biofilm, that were responsible for recalcitrance, discussed in detail below, wherein once the antibiotic concentration decreased, the persister sub-population would repopulate the biofilm. See e.g., Lewis, K., Riddle of biofilm resistance, Antimicrobial Agents and Chemotherapy, 45(4):999-1007 (2001), incoprated by reference herein.

Persisters are dormant variants of regular cells that form stochastically in microbial populations and are highly tolerant to microbicides. Persisters play a major role in the recalcitrance of chronic infections to antibiotics. While they were discovered nearly 80 years by Colonel Joseph Bigger in 1944 of Trinity College (Dublin, Ireland) who published a paper in The Lancet that reported on two important discoveries penicillin is a tidal rather than a bacteriostatic antibiotic, contrary to the prevailing opinion at the time and treatment of a population of staphylococci with penicillin failed to sterilize the culture, leaving a small portion of cells that he named “persisters”. Bigger considered two main hypotheses: (i) persisters have a higher heritable resistance to growth inhibition by penicillin, and (ii) persisters are variants that have the same susceptibility to growth inhibition by penicillin as the bulk of the cells but are insensitive to killing by penicillin. Bigger showed that upon regrowth, persisters that survived treatment with penicillin produce populations indistinguishable from the original strain and persisters they are similarly sensitive to growth inhibition and produce new persisters. High persister (hip) mutants of Pseudomonas aeruginosa are selected in patients with cystic fibrosis. Similarly, hip mutants of Candida albicans are selected in patients with an oral thrush biofilm. These observations suggest that persisters may be the main culprit responsible for the recalcitrance of chronic infectious disease to microbicide or therapy. Targeting persisters holds the promise of effectively treating chronic infections. See e.g., Bigger, J. W., Treatment of staphylococcal infections with penicillin. Lancet ii:497-500 (1944), incorporated by reference herein.

Thus, persister cells, referered to herein generaly as persisters, are a continually re-occuring sub-population located in a microbil biofilm which can survive microbicides by being going into a state of dormancy. Such a state of domancy may be a produced by highly regulated and sophisticated regulatory mechanism of gene expression. It is theorized that microbil biofilm resistance is based on persister survival resulting in reoccuring infections.

Antimicrobial resistance is one of the most important microbial processes and widely recognized as a major public health threat worldwide. Multidrug resistant organisms, resistant to microbicides, several including classes of antibiotic, are present not only in hospital environments but also identified in community settings, thus establishing resistant reservoirs that can present themselves anywhere. Bacterial response to microbicides through a variety of adaptation mechanisms as a consequence of their high genetic variability, growth rates, and population size and includes mutational adaptations, acquisition of genetic material or alteration of gene expression which has produced demonstratable resistance to virtually all microbicides in clinical practice. Understanding the biochemical and genetic basis of resistance is critical towards design of strategies that confront the spread of antimicrobial resistance to microbicides and innovative therapies are critical as the world approaches greater frequencies of multidrug-resistant organisms, See e.g., Antimicrobial Resistance: Global Report on Surveillance World Health Organization (2014), and Antibiotic Resistance Threats in the United States. Centers for Disease control and Prevention (2013), both, incorporated by reference herein.

### SUMMARY OF THE INVENTION

In one aspect, a system to eradicate a majority of disease-causing germs and produce avirulent phenotype survivors susceptible to microbicides using a light dose regiment is featured. The system includes one or more processors and one or more light sources coupled to the one or more processors. The one or more processors are configured to control the one or more light sources to apply the light dose regiment including a) continuously applying a pre-treatment of non-lethal UVA light to the disease-causing germs to increase the production of pigment to the disease-causing germs, b) simultaneously applying pulsed UVA light and continuous UVC light to the disease-causing germs having the increased pigment therein to eradicate the majority of the disease-causing germs, and c) cycling steps a) and b) a selected number of times to generate avirulent phenotype survivors susceptible to microbicides.

In one embodiment, the cycling steps a) and b) the selected number of times to generate the avirulent phenotype survivors susceptible to rnicrobicides may be based on the number of disease-causing germs and the distance the disease-causing germs are from the one or more light sources. The light dose regiment may include a number of times steps a) and b) are performed. The characteristics of the light dose regiment may include one or more of: a wavelength of the UVA light and/or a wavelength of the UVC light, a wavelength of waveform of the UVA light and/or a waveform of the UVC light, a light power of the UVA light and/or a light power UVC light and/or an amount of time each of steps a) and b) are performed. The germs may include one or more of disease-causing bacteria and fungi. The microbicides may include one or more of: antibiotics, antifungal agents, and antiseptics, The avirulent phenotype survivor susceptible to microbicides may include disease-causing bacteria and fungi. Cycling steps a) and b) the selected number of times may produce the avirulent phenotype survivors susceptible to microbicides which are stable over multiple generations. Cycling steps a) and b) the selected number of times may produce the avirulent phenotype survivors susceptible to microbicides which are unable to produce a microbial biofilm associated with reoccurring infections. Cycling steps a) and b) the selected number of times may convert persisters in a microbial biofilm associated with reoccurring infections from a dormant state to a non-dormant state thereby making the persisters susceptible to microbicides. The cycling steps a) and b) the selected number of times may disrupt gene regulation, transcription, and translation yielding an avirulent survivors phenotype that is characterized by a permanently and genetically altered state unable to maintain normal physiological gene regulation, including production of virulence factors, and germicidal resistance mechanisms to microbicides thereby rendering the avirulent phenotype survivors susceptibility to microbicides and non-infectious. Cycling steps a) and b) the selected number of times may be performed on the skin or soft tissue of a human subject or an animal. Cycling steps a) and b) the selected number of times may be performed on an inert surface and may be combined with the microbicides to eradicate a majority of the disease-causing germs on the inert surface. The avirulent germicide susceptible phenotype survivors susceptibility to germicides remains even after multiple generations. Cycling steps a) and b) the selected number of times to generate avirulent phenotype survivors susceptible to microbicides may rescue failed microbicides and/or improves the effectiveness of microbicides.

In another aspect, a method to eradicate a majority of disease-causing germs and produce avirulent phenotype survivors susceptible to microbicides with a light dose regiment is featured. The method includes a) continuously applying a pre-treatment of non-lethal UVA light to the disease-causing germs to increase the production of pigment to the disease-causing germs, b) simultaneously applying pulsed UVA light and continuous UVC light to the disease-causing germs having the increased pigment therein to eradicate the majority of the disease-causing germs, and c) cycling steps a) and b) selected number of times to generate avirulent phenotype survivors susceptible to microbicides.

In one embodiment, cycling steps a) and b) the selected number of times to generate the avirulent phenotype survivors susceptible to microbicides may be based on the number of disease-causing germs and the distance the disease-causing germs are from the one or more light sources, The light dose regiment may include a predetermined number of times steps a) and b) are performed. The characteristics of the light dose regiment may include one or more of: a wavelength of the UVA light and/or a wavelength of the UVC light, a waveform of the UVA light and/or a waveform of the UVC light, a light power of the UVA light and/or a light power of the UVC light and/or an amount of time each of steps a) and b) are performed. The germs may include one or more of disease-causing bacteria and fungi. The microbicides may include one or more of: antibiotics, antifungal agents, and antiseptics. The avirulent phenotype survivors susceptible to microbicides may include disease-causing bacteria and fungi. Cycling steps a) and b) the selected number of times may produce the avirulent phenotype survivors susceptible to microbicides which are stable over multiple generations. Cycling steps a) and b) the selected number of times may produce the avirulent phenotype survivors susceptible to microbicides which are unable to produce a microbial bicifilm associated with reoccurring infections. Cycling steps a) and b) the selected number of times may convert persisters in a microbial biofilm associated with reoccurring infections from a dormant state to a non-dormant state thereby making the persisters susceptible to microbicides. Cycling steps a) and b) the selected number of times may disrupt gene regulation, transcription, and translation yielding a avirulent phenotype survivors phenotype which permanently and genetically alters the virulent phenotype survivors phenotype to produce the avirulent phenotype survivors susceptible to microbicides and renders the avirulent phenotype survivors unable to maintain normal physiological function including the regulation and production of virulence factors and increases the avirulent phenotype survivors susceptibility to microbicides. Cycling steps a) and b) the selected number of times may be performed on the skin or soft tissue of a human subject or an animal. Cycling steps a) and b) the selected number of times may be performed on an inert surface and may be combined with the microbicides to eradicate a majority of the disease-causing germs on the inert surface. The avirulent germicide susceptible phenotype survivors may be stable over multiple generations. Cycling steps a) and b) the selected number of times to generate avirulent phenotype survivors susceptible to microbicides may rescue failed microbicides and/or improves the effectiveness of microbicides.

The subject invention, however, in other embodiments, need not achieve all these objectives and the claims hereof should not be limited to structures or methods capable of achieving these objectives.

### BRIEF DESCRIPTION OF THE SEVERAL VIEWS OF THE DRAWINGS

Other objects, features and advantages will occur to those skilled in the art from the following description of a preferred embodiment and the accompanying drawings, in which:

FIG. 1 is schematic block diagram showing the primary components of one embodiment of the system to eradicate a majority of disease-causing germs and produce avirulent phenotype survivors susceptible to microbicides using a light dose regiment;

FIG. 2 is a block diagram showing one example of the steps of the method to eradicate a majority of disease-causing germs and produce avirulent phenotype survivors susceptible to microbicides using a light dose regiment;

FIG. 3A and 3B show example growth curves of selected avirulent phenotype survivors susceptible to microbicides for the system and method shown in FIG. 1 and 2 after treatment with the light dose regiment;

FIG. 4 shows an example of control growth of S. aureus before exposure to the light dose regiment of the system and method shown in FIG. 1 and 2;

FIG. 5 shows an example of the S. aureus shown in FIG. 4 after cycling steps a) and b) for the system and method shown in FIGS. 1 and 2 one time using the light dose regiment wherein the S. aureus is partially depigmented;

FIG. 6 shows an example of the S. aureus shown in FIG. 4 after a light dose regiment applied by the system and method shown in FIGS. 1 and 2 cycled one time showing the S. aureus completely depigmented to produce avirulent survivors susceptible to microbial agents;

FIG. 7 shows examples of Kirby-Bower zones of inhibition (ZOI) assays used to determine the lowest concentration of antimicrobial agents that prevents visible growth for a microorganism for two consecutive generations of selected avirulent phenotype survivors susceptible to microbicides;

FIG. 8 shows heat map profiles of one examples of the resistance and susceptibility of S. aureus Parental Wild Type to various antibiotics compared to depigmented generation 1 of an avirulent phenotype survivors susceptible to microbicides and compared to Generation 2 of a depigmented avirulent phenotype survivors susceptible to microbicides;

FIG. 9A and 9B show an example of a quadrant streak performed on sheep blood agar used to compare MRSA wild type strain to a Generation 2 avirulent phenotype survivors susceptible to microbicides created using the light dose regiment of the system and method shown in FIGS. 1 and 2;

FIG. 10A shows an example of the contribution of S. aureus Staphyloxanthin to virulence and in vivo by direct comparison of an isogenic pair of wild type with a knock out nonpigmented mutant strain;

FIG. 10B depicts an example of a representative mouse from the corresponding challenge groups in FIG. 10A.;

FIG. 11 shows an example of S. aureus parental wild type, a depigmented survivor first generation avirulent phenotype survivor susceptible to microbicides, and a depigmented survivor second generation avirulent phenotype survivor susceptible to microbicides;

FIG. 12 is a schematic block diagram showing an example of the genetic deletion of a Carotenoid pigment pathway to eliminate the production of S. aureus;

FIG. 13 shows a schematic illustration of virulence photoactivation provided by the light dose regiment of the system and method shown in one or more of FIGS. 1-12;

FIG. 14 is a schematic illustration showing examples of four mechanisms used by disease-causing germs to prevent their susceptibility to microbicides;

FIG. 15 is a schematic illustration showing examples of key members of the five super-families of the microbial efflux system shown in FIG. 14;

FIG. 16 shows a schematic illustration of a model of time-lapse microscopy experiment and a model of how ppGpp stochastically induces persistence in E. coli;

FIG. 17 is a schematic illustration showing an example of biofilm resistance based on persister survival;

FIG. 18A depicts an example of persisters surviving in a biofilm treatment with an antibiotic;

FIG. 18B depicts an example of planktonic cells treated with an antibiotic showing the absence of persisters;

FIGS. 19A, 19B, and 19C show plots showing sample distances comparing a control to depigmented generation 1 avirulent phenotype survivors, the control to depigmented generation 2 avirulent phenotype survivors, and the sample distance between depigmented generation 1 avirulent phenotype survivors to depigmented generation 2 avirulent phenotype survivors;

FIG. 20 shows plots 20A, 20B, and 20C show examples of principal component analysis of a control parental wild type to depigmented generation 1 avirulent phenotype survivors to the control parental wild type to depigmented generation 2 avirulent phenotype survivors, and depigmented generation 1 avirulent phenotype survivors to depigrnented generation 2 avirulent phenotype survivors;

FIGS. 21A, 21B, and 21C show examples of heat maps of differently expressed genes of the S. aureus parental wild type control to depigmented generation 1 avirulent phenotype survivors, to depigmented generation 2 avirulent phenotype survivors, and depigmented generation 1 avirulent phenotype survivors to depigmented generation 2 avirulent phenotype survivors; and

FIG. 22 is a graph showing an example of the lower light energy of UVA light and UVC light needed by the system and method shown in one or more of FIGS. 1-21.

### DETAILED DESCRIPTION OF THE INVENTION

Aside from the preferred embodiment or embodiments disclosed below, this invention is capable of other embodiments and of being practiced or being carried out in various ways. Thus, it is to be understood that the invention is not limited in its application to the details of construction and the arrangements of components set forth in the following description or illustrated in the drawings. If only one embodiment is described herein, the claims hereof are not to be limited to that embodiment. Moreover, the claims hereof are not to be read restrictively unless there is clear and convincing evidence manifesting a certain exclusion, restriction, or disclaimer.

Virulence factors are molecules produced by disease-causing germs which increase their effectiveness and enable them to thrive and survive. Virulence factors include, inter alia, colonization in the host, evasion of the host immune response, inhibition of the host immune response, and transport through the cell wall of disease-causing germs. Some disease-causing germs include a wide array of virulence factors some of which are chromosomally encoded into the disease-causing germs and others may be obtained from mobile genetic elements such as plasmids and bacteriophages. Virulence factors encoded on mobile genetic elements spread through gene transfer and convert harmless bacteria into dangerous disease-causing germs.

Pigmentation is a common de facto virulence factor that operates by selectively absorbing light. The major virulence factor attributed with microbial pigments is the interference with innate and adoptive host pathways enhancing cytotoxicity or by showing pro-inflammatory properties. Pigments are central in the metabolic regulation of disease-causing germs and a multitude of protective properties. Examples of virulence factors are discussed below.

TABLE 1
Selected Examples of S. aureus Virulence Factors

| Trait | Genes encoding selected factors | Associated infections |
|---|---|---|
| Attachment | c [symbol] fA, c [symbol] fB: clumping factors; [symbol] : libronectin-binding proteins: cna, acm, ace [symbol] , | skin lesions in atopic |
| | collagen-binding adhesins (S. aureus and homologues in E. faecalis, E. faecium); [symbol] sdA: | dermatitis, abscesses, |
| | iron-regulated surface protein A; sdr: adherence protein Sdr; [symbol] | necrotic skin lesions |
| | formation | chronic and biofilm- |
| Persistence | hemB: fibrinogen-binding protein; srtA, bps (SrfC): sortases; opp-3: ABC transporter in | associated skin |
| | staphylococcai species; lasA, lasB, rhlA [symbol] ; Transcriptional regulator for LasRI system | infections |
| | and Rhi Quorum Sensing systems; SasX: S. aureus surface protein X; SasC, SasB, SasD, | |
| | SasF, SasJ, SasK, SasL; attachment and accumulation in biofilm formation; [symbol] | |
| | excopolysaccharide biosynthesis; ompR/env [symbol] ; osmoregulation and expression of outer | |
| | membrane porine | |
| Evading/ | lukS-PV, lukF-PV: cytotoxin Panton-Valentine leuccocidin complex; [symbol] | invasive skin |
| destroying | gamma-hemolysin; [symbol] cell surface-associated extracellular adherence proteins; exoS, | infections, abscesses, |
| host defense | [symbol] biofilm production in P. aeruginosa; [symbol] secondary arginine deiminase system [symbol] | diabetic foot infections |
| | [symbol] encodes a potassium transporter system; [symbol] | |
| | operons and genes implicated in pyocyanin production; pchB [symbol] | |
| | biosynthesis; [symbol] protein A | |
| Tissue | hysA: encoding the exoenzyme hyaiuronidase; [symbol] proteases; [symbol] alpha toxin; [symbol] | destructive metastatic |
| invasion | serine protease SepA autotransporter; [symbol] filament component; [symbol] | soft tissue infections, |
| | specific ATP synthase: secA, secD, secE, SecG, secY; protein translocation | diabetic foot infections |
| Toxins | [symbol] toxic shock syndrome toxin; eta, [symbol] staphylococcal exfoliative toxins A and B; toxA, | toxic shock, scalded |
| | [symbol] involved inType III Secretion System; | skin, impetigo, burn |
| | sagA; encodes peptide toxin [symbol] S | wound infections, |
| | | necrotizing fasciitis |
| Pigments | Staphyloxanthin C₃₀ triterpenoid carotenoid biosynthesies pathway, crt [symbol] controlled | |
| | by the alternative sigma factor SigB. | |

[symbol]

indicates data missing or illegible when filed

Expression and regulation for most of the virulence factors differ according the growth phase and the environmental and culture conditions. The main regulators are either proteins associated with the cell wall or secreted toxins that function accordingly. Most of the classes of virulence factors operate by triggering different genes or clusters. Gene expression is regulated by specific and sensitive mechanisms at the transcriptional level. Regulatory factors are compiling complex networks and drive specific interactions with target gene promoters. These factors are largely regulated by two-component regulatory systems, such as the agr, saeRS, srrAB, arlSR, lytRS systems, and the like. These systems are sensitive to environmental signals and consist of a sensor histidine kinase and a response regulator protein. DNA-binding proteins, such as SarA and the recently identified SarA homologues, e.g., SarR, Rot, SarS, SarT, SarU, also regulate virulence factor expression. These homologues may be intermediates in the regulatory networks. The multiple pathways generated by these factors allow the bacterium to adapt to environmental conditions rapidly and specifically, and to develop infection. An example of the consortium of genes and operons regulated by the agr two-component system is provided in Table 2 below. See e.g., Bronner et al, Regulation of Virulence Determinants in Staphylococcus aureus: Complexity And Applications, FEMS Microbiology Reviews, Volume 28, Issue 2, (2004), incorporated by reference herein.

TABLE 2
Gene Regulation by agr

| Protein name | Gene | Effect |
|---|---|---|
| Capsular | cap5 | + |
| polysaccharide | | |
| (type 5) | | |
| Capsular | cap8 | + |
| polysaccharide | | |
| (type 8) | | |
| Protein A | spa | − |
| Fibronectin- | fnbA | − |
| binding | fnbB | − |
| protein | | |
| Alpha-toxin | hla | + |
| Beta- | hlb | + |
| hemolysin | | |
| Delta- | hld | + |
| hemolysin | | |
| Gamma- | hlgA | + |
| hemolysin | | |
| Panton- | luk- | + |
| Valentine | PV | |
| Leucocidin | | |
| LukE- | lukED | + |
| LukD | | |
| TSST-1 | tst | + |
| Enterotoxin | seb | + |
| B | | |
| Enterotoxin | sed | + |
| C | | |
| Enterotoxin | sed | + |
| D | | |
| Exfoliatin A | eta, | + |
| and B | etb | |
| V8 serine | aspA | + |
| protease | | |
| Proteases, | slpA, | + |
| E, F | B, C | |
| | D, E, | |
| | F | |

Many pathogens appear a distinctive colony color when they propagate under laboratory conditions. Pigmentation is a common de facto virulence factor that operates by selectively absorbing light. The biochemistry and genetics of pigments identifies a variety of structures present in a broad range of pathogens. See Table 3 below. See Liu, G., & Nizet, V., Color Me Bad: Microbial Pigments As Virulence Factors, Trends Microbiol, 17(9): 406-413 (2009) and Pelt et al., Structure and Biosynthesis of Staphyloxanthin From Staphylococcus Aureus, J. Biol. Chem., 280:32493-32498 (2005), both incorporated by reference herein. The major virulence attribute of microbial pigments is interference in the innate and adoptive host immune pathways, enhancing cytotoxicity, or by showing pro-inflarrimatory properties. Pigments are central in metabolic regulation, e.g., iron, nutrients, and energy acquisition, and a multitude of protective properties, e.g., reduce the susceptibility of antimicrobials or exhibiting antimicrobial properties against other pathogens or competitors, and protect against physical and environmental conditions, e.g., heat temperature ultraviolet radiation. The major pigment of S. aureus is the carotenoid Staphyloxanthin. Biosynthesis of Staphyloxanthin is regulated by, crtOPQMN, with a σ⁸-dependent promoter upstream of crtO and a termination region downstream of crtN.

TABLE 3
Properties of Microbial Pigments

| Pigment | Chemistry | Color | Human pathogens | Virulence functions |
|---|---|---|---|---|
| Staphyloxanthin | Carotenoid | Golden | Staphylococcus aureus | Antioxidant, detoxify |
| | | | | ROS |
| Pyocyanin | Phenazine-derived | Blue green | Pseudomonas spp. | Cytotoxicity |
| | zwitterion | | | Neutrophil apoptosis |
| | | | | Ciliary dysmotility |
| | | | | Proinflammatory |
| Melanin | Polyacetylene or | Dark | Cryptococcus neoformans, | Antioxidant |
| | polypyrrine polymers | brown, | Aspergillus spp., Wangiella | Antiphagocytic |
| | | black | dermatitidis, Sporothrix schenckii, | Block antimicrobials |
| | | | Burkholderia capacia | |
| Porphyrin | Heteromacrocycte | Black | Porphyromonas gingivalis | Antioxidant, detoxify |
| | | | | ROS |
| Granadaene | Ornithine | Orange red | Streptococcus agalactiae | Antioxidant, detoxify |
| | rhamnopolyene | | | ROS |
| Violacein | Rearranged | Purple | Chromobacterium violaceum | Antioxidant, detoxify |
| | pyrrolidone scaffold | | | ROS |
| Prodigiosin | Linear tripyrrole | Red | Serratia marcescens | Immunosuppressant |
| Hemozoin | β-hematin | Brown | Plasmodium spp. | Detoxification |
| | aggregates | black | | Macrophage |
| | | | | suppression |
| | | | | Pro-inflammatory |

The biofilms discussed in the Background section above may be representative of phenotypic resistance and may include multidimensional polymicrobial communities thriving within a protected network over antimicrobials and host defense mechanisms. Polymicrobial bitafilms prevail over naono-species in the wound microenvironment and build up bacterial phenotypes resilient to eradication. The complex biofilm hosts the metabolically quiescent and antibiotic-tolerant phenotypes and the slow-growing small-colony variants (SCVs) that puzzle diagnosis and treatment by avoiding detection.

Specialized non-dividing survivor cells, the persisters discussed in the Background section above, protecting bacterial populations from antibiotic killing is also a potential route to antibacterial therapies. Persisters may survive challenge by microbicides by virtue of their metabolic inactivity. Eradication of these cells is currently challenging because the exact mechanisms of persister formation are still unclear. The following references support the discussion above regarding virulence factors, biofilms, and persisters: Krishnan N., We Avoid Antibiotic Lock Solutions Due to Fear of Antibiotic Resistance, Semin Dia, 29:289-91 (2016); Chopra V, et al, PICC-Associated Bloodstream Ifections: Prevalence, Patterns, and Predictors, Am J. Med,127: 319-28 (2014); Murray C. et al., Infections Complicating The Care of Combat Casualties During Operations Iraqi Freedom and Enduring Freedom, J Trauma, 71(1 Suppl);S6273 (2011); Wood P. et al, Clinical and Microbiological Evaluation of Epidural and Regional Anaesthesia Catheters in Injured UK Military Personnel, J. R. Army Med Corps. (2015); O'Grady, N. et al., Guidelines for the Prevention of Intravascular Catheter-Related Infections, Clinical Infectious Diseases, 52:1087-99 (2011); Buetti, N. et al., Does The Spectrum of Microbial Causes Change Over Time? A Nationwide Surveillance Study, Swiss Centre for Antibiotic Resistance (ANRESIS), Catheter-Related Infections, BMJ Open, 8:e023824 (2015); Basil A. et al., Epidemiological, Demographic, and Outcome Characteristics of Burn Injury, in Total Burn Care, Saunders Elsevier: Philadelphia, Pa. (2007); Renz et al., Acute Burn Care, in Combat Casualty Care-Lessons Learned from QEF and OIF, Office of the Surgeon General, Borden Institute: Fort Detrick, Md. (2012); Haberal M. et al., Fluid Management in Major Burn Injuries, Indian J Plast Surgery, 43 (Suppl):529-36 (2010); Eastride B J et al., Death on the Battlefield (2001-2011): Implications for the Future of Combat Casualty Care, The Journal of Trauma and Acute Care Surgery, 73(6 Suppl 5): p. S431-7;S431-7 (2012); Lai T, et al Continuous Peripheral Nerve Block Catheter Infections in Combat-Related Injuries: a Case Report of Five Soldiers from Operation Enduring Freedom/Operation Iraqi Freedom, Pain Medicine, 12:1676-81 (2011); Hulse et al., Vascular Access on the 21st Century Military Battlefield, J. R. Army Med Corps, 156(4 Suppl 1): p, 385-90, :385-90 (2010); O'Grady Nea, Guidelines for the Prevention of Intravascular Catheter-Related Infections, Clin Infect Dis., 52:e162-93 (2011); Nakazawa N. Infectious and Thrombotic Complications of Central Venous Catheters, Semin ° flea) Nurs., 26;121-31 (2010); C.D.C., National and State Healthcare-Associated Infections Progress Report, available at https://wwwcdcgov/hai/data/portal/progress-reporthtml (2018); O'Grady et Guidelines for the Prevention of Intravascular Catheter-related Infections, Clinical Infectious Diseases, 52:1087-99 (2011); Buetti et at, Catheter-Related Infections: Does the Spectrum of Microbial Causes Change Over Time? A Nationwide Surveillance Study, BMJ Open, 8:e023824 (2018); Hutchens et al., Calibration of Optimal Use Parameters for an Ultraviolet Light-Emitting Diode in Eliminating Bacterial Contamination on Needieless Connectors, J Appl Microbiol., 118:1298-305 (2015); Jarvis et al., Health Care-Associated Bloodstream Infections Associated With Negative or Positive-Pressure or Displacement Mechanical Valve Needleless Connectors, Clin. Infect. Dis., 49:1821-7 (2009); Hadaway L., Needleless Connectors for IV Catheters, Am. J. Nurs., 112:32 (2012); Crnich C et al., The Promise of Novel Technology for the Prevention off. Intravascular Device-Related Bloodstream Infection, II. Long-Term Devices, Clin Infect Dis. 34:1362-8 (2002): Mermel L, What is the Predominant Source of Intravascular Catheter Infections? Clin Infect Dis. 52:211-2 (2011); Bouza et al., A Needleless Closed System Device (CLAVE) Protects from Intravascular Catheter Tip and Hub Colonization: a Prospective Randomized Study, J. Hosp. Infer 54(4):279-87. PubMed PMID: 12919758 (2003); Field et al., Incidence of Catheter-Related Bloodstream Infection Among Patients With a Needleless, Mechanical Valve-Based Intravenous Connector in an Australian Hematology-Oncology Unit, Infect. Control Hosp. Epidemiol, 28:610-3 (2007); Rupp et al., Outbreak of Bloodstream Infection Temporally Associated With the Use of an Intravascular Needleless Valve, Clin. Infect. Dis. 244:1408-14 (2007); Holroyd et al Universal Intravenous Access Cleaning Device Fails to Sterilize Stopcocks, Anesth, Analg. 118:333-43 (2014); Mourcau et al., Disinfection of Needleless Connector Hubs Clinical Evidence Systematic Review, Nursing Research and Practice. 796762, doi: 10.1155/20151796762, PubMed PMID 26075093; PMCID: 4446481 (2015); O'Grady et al, Summary of Recommendations: Guidelines for the Prevention of Intravascular Catheter-Related Infections, Clin. Infect. Dis. 52:1087-99 (2011); Hadaway L., Needleless Connectors for IV Catheters, Am. J. Nurs. 112(11)3244. doi: 10.1097/01.NAJ. 0000422253.72836.cl. PubMed 23075549 (2012); Karchmer et al., editors. Needleless Valve Ports May Be Associated With a High Rate of Catheter-Related Bloodstream Infection, Program and Abstracts of the 15th Annual Scientific Meeting of the Society for Healthcare Epidemiologists of America (2005); Simmons et al., Scrub the Hub: Cleaning Duration and Reduction in Bacterial Load on Central Venous Catheters. Crit. Care Nurs. Q. 34:31-5 (2011); Mahieu et al., Catheter Manipulations and the Risk of Catheter-Associated Bloodstream Infection in Neonatal Intensive Care Unit Patients, J. Hosp. Infect. 48:2163 (2001); Holroyd et al., Incidence of Central Venous Catheter Hub Contamination, Journal of Critical Care. 39;162-8, doi: 10.1016/j.jcrc,2017.02.035. PubMed PMID: 28259730 (2017); Liu et al., Staphylococcus Aureus Golden Pigment Impairs Neutrophil Killing and Promotes Virulence Through its Antioxidant Activity, J. Exp. Med. 202:209-15; (2005); Hassan et al., Inactivation of the GarA Response Regulator in Pseudomonas Fluorescens Pf-5 Has Far-Reaching Transcriptomic Consequences, Environ Microbial., 2(4):899-915. Epub 2010/01/22, doi: EM12134 [pii] 10.1111/j.1462-2920.2009.02134.x. PubMed PMID: 20089046 (2010); Lim et al., The Effect of Zinc Limitation on the Transcriptoine of Pseudomonas Protegens Pf-5, Environ Microbial., Epub 2012/08/21, dol: 10.1111/j.1462-2920.2012.02849.x. PubMed PMID; 22900619 (2012); Lim et al, The Effect of Iron Limitation on the Transcriptome and Proteome aaf Pseudomonos Fluorescens Pf-5, PloS one, 7(6):e39139, Epub 2012/06/23, doi: 10.1371/journal.pone.0039139 PONE-D-12-02073 [pii]. PubMed PMID: 22723948 (2012); Trapnell et al., Differential Gene and Transcript Expression Analysis of RNA-seg Experiments With TopHat and Cufflinks, Nat Protoc. 7(3):562-78, Epub 2012/03/03, doi: nprot.2012.016 [pii] 10.1038/nprot,2012,016, PubMed PMID: 22383036 (2012); Passalacqua et al., Comparative Transcriptional Profiling of Bacillus Cereus Sensu Lato Strains During Growth in CO2-bicarbonate and Aerobic Atmospheres, PloS one, 4(3):e4904. Epub 2009/03/20. doi: 10.1371/journal.pone, 0004904, PubMed PMID; 19295911 (2009); Mohr et al., Inactivation of Pathogens in Platelet Concentrates By Using a Two-Step Procedure, Vox Sang, 84:96-104 (2003); Faergemann et al., The Effect of UV-Light on Human Skin Microorganisms, Acta. Derm. Venereal., 67:69-72 (1987); Tingpej et al., Decontamination Efficacy of Ultraviolet Radiation against Biofibus of Common Nosocomial Bacteria. J. Med. Assoc. Thai., 98:5828 (2015); El-Azizi et al., Efficacy of Ultraviolet C Light at Sublethal Dose in Combination With Antistaphyloroccal Antibiotics to Disinfect Catheter Biofilms of Methicillin-Susceptible and Methicillin-Resistant Staphylococcus Aureus and Staphylococcus Epidermidis in vitro. Infect. Drug Resist. 19:181-9 (2016); Shoults et al., Total Staphylococci as Pe ormance Surrogate For Greywater Treatment, Environ. Pollut. Res. Int. 33:32894-900 (2018); Nakamura H., Sterilization Efficacy of Ultraviolet Irradiation on Microbial Aerosols Under Dynamic Airflow by Experimental Air Conditioning Systems, Bull Tokyo Med Dent Univ., 34:25-40 (1987); Shoults et al., Decreased Efficacy of UV Inactivation of Staphylococcus Aureus After Multiple Exposure and Growth Cycles. Int. J. Hyg. Environ. Health, 222:111-6 (2019); Guillobel et al., Characterization of Staphylococcus Epidermidis Mutants Sensitive to Ultraviolet Radiation, Mutat. Res. 193;1-10 (1988); Tyrrell et al., Interactions Between UV Radiation of Different Energies in the Inactivation of Bacteria. J. Bacteriol. 136;437-40 (1978); Wengraitis et al., Pulsed UV-C Disinfection of Escherichia Coli With Light-Emitting Diodes, Emitted at Various Repetition Rates and Duty Cycles, Photochem. Photobiol. 89:127-31 (2013); Li et al., Enhanced Germicidal Effects of Pulsed UV-LED Irradiation on Biofilms, J. Appl. Micrabiol., 109:2183-90 (2010); Dong et al., Photolysis of Staphyloxanthin in Methicillin-Resistant Staphylococcus aureus Potentiates Killing by Reactive Oxygen Species, Adv. Sri, (\Weinh). 6:1900030 (2019); Tsai et al. The Group A Streptococcus Serotype M2 Pilus Plays a Role in Host Cell Adhesion and Immune Evasion, Molecular Microbiology, 103:282-98 (2017); Jesse et al., Front. Microbiol 8, 2433, doi:10,3389/fmicb.2017.02433 (2017), Staphylococcal Adhesion and Host Cell Invasion: Fibronectin-Binding and Other Mechanisms, Front Microbiol. 2017;8:2433 (2017); Malachowa Nea, Contribution of Staphylococcus Aureus Coagtelases and Clumping Factor A to Abscess Formation in a Rabbit Model of Skin and Soft Tissue Infection, PloS one. 11;e0158293 (2016); Foster et al., Adhesion, Invasion and Evasion: the Many Functions of the Surface Proteins of Staphylococcus Aureus, Nat. Rev. Microbial, 12:49-62 (2014); O'Connor et at, First Description of Novel Arginine Catabolic Mobile Elements (ACMEs) Types IV and V Harboring a kdp Operon in Staphylococcus Epidermidis Characterized by Whole Genome Sequencing, Infection, genetics and evolution. Journal of Molecular Epidemiology and Evolutionary Genetics in Infectious Diseases, 61:60-6 (2018); Tipton KAR, P. N. An ompR/envZ Two-Component System Ortholog Regulates Phase Variation, Osmotic Tolerance, Motility, and Virulence in Acinetobacter baumannii strain, AB5075. J. Bacteriol. doi:10.1128/JB.00705-16 (2016); Meleharti et al, Staphylococcus Aureus Leukocidin A/B (LukAB) Kills Human Monocytes Via Host NLRP3 and ASC When Extracelhdar, But Not Intracellular. PLoS pathogens, 11:e1004970 (2015); Shallcross et al., The Role of the Panton-Valentine Leucocidin Toxin in Staphylococcal Disease: A Systematic Review and Meta-Analysis, The Lancet Infectious Diseases, 13:43-54 (2013); Botka T. Two Highly Divergent Lineages of Exfoliative Toxin B-Encoding Plasmids Revealed in Impetigo Strains of Staphylococcus Aureus International Journal of Medical Microbiology, IJMM 307, 291-296 307:291-6 (2017); Flaherty et al., Streptolysin S Promotes Programmed Cell Death and Enhances In ammatory Signaling in Epithelial Keratinocytes daring Group A Streptococcus Infection. Infection and Immunity, 83:4118-33 (2015); Bronner et al Regulation of Virulence Determinants in Staphylococcus Aureas: Complexity and Applications, FEMS Microbiology Reviews, 28:183-200 (2004); Liu et al., Color Me Bad: Microbial Pigments as Virulence Factors Trends Microbial., 17:406-13 (2009); Pelz et al., Structure and Biosynthesis of Staphyloxanthin From Staphylococcus Aureas, J. Biol, Chem. 280:32493-8 (2005); Magana et al., Options and Limitations in Clinical Investigation of Bacterial Biofilms, Clin. Microbiol, Rev. 31 (2018); Lechner et al., Staphylococcus Aureus Persisters Tolerant to Bactericidal Antibiotics, J. Mol. Microbiol. Biotechnol. 32:23544 (2012). All of the above references are incorporated by reference herein.

The inventor hereof discovered that exposing disease-causing germs to a pretreatment of a non-lethal dose of UVA light increases the production of pigment in disease-causing germs. The inventor also discovered that exposing the disease-causing germs with the increased pigmentation therein to an innovative combination of UVA light and UVC light having specific waveforms, wavelengths and light power eradicates a majority of the disease-causing germs and the cyclobutane dimers produced from the UVC light will cause low fidelity translation of DNA which will leave a small population of disease-causing germs mutants that are avirulent and susceptible to antibiotics. Then, if the pre-treatment step and the exposure to the novel combination of UVA light and UVC light are cycled or repeated as a light dose regiment, a select number of times, avirulent phenotype survivors susceptible to microhicides are produced. The pretreatment step and the exposure to the novel cycled combination of UVA light and UVC light also produces avirulent phenotype survivors susceptible to microbicides which are unable to produce a microbial biofilm associated with reoccurring infections, converts persisters in a microbial biofilm associated with reoccurring infections from a dormant state to a non-dormant state thereby making the persisters susceptible to microbicides and interferes with gene regulation, transcription, and translation yielding an avirulent survivors phenotype that is characterized by a permanently and genetically altered state unable to maintain normal physiological gene regulation, including production of virulence factors, and germicidal resistance mechanisms to microbicides thereby rendering the avirulent phenotype survivors susceptibility to microbicides and non-infectious.

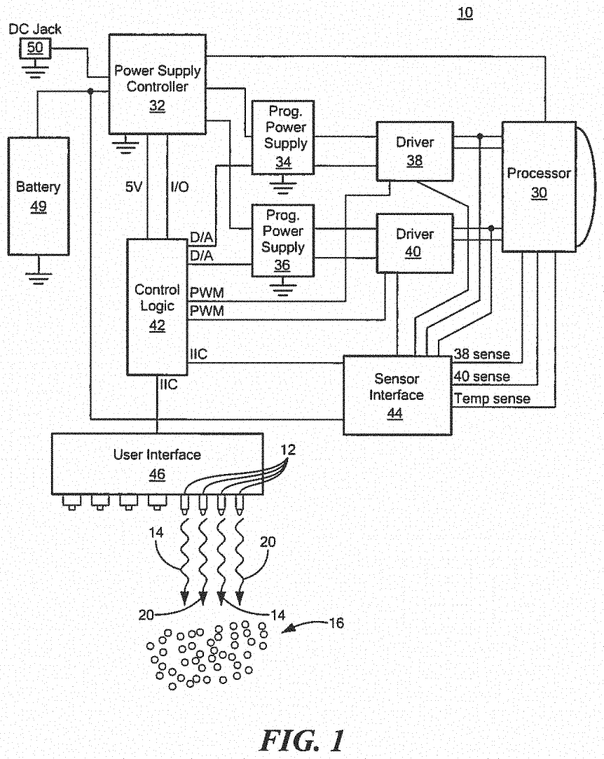

There is shown in FIG. 1 one embodiment of system 10 for eradicating a majority of disease-causing germs and produce avirulem phenotype survivors susceptible to microbicides using a light dose regiment. System 10 include one or more light sources 12, e.g., light emitting diodes (LEDs) or similar type light sources coupled to processor 30. Processor 30 may include one or more processors, an application-specific integrated circuit (ASIC), firmware, hardware, and/or software (including firmware, resident software, micro-code, and the like) or a combination of both hardware and software that may all generally be referred to herein as a “processor”. In one example, processor 30 may be integrated into a Multi Emitter Package (MEP). Processor 30 is coupled to one or more light sources 12 and controls the one or more light sources 12 to perform step a): continuously apply a pre-treatment of non-lethal UVA light 14 to disease-causing germs 16 to increase the production of pigment in disease-causing germs 16. Processor 30 also controls the one or more light sources 12 to perform step b): simultaneously apply pulsed UVA light 14 and continuously apply UVC light 20 to disease-causing germs 16 having the increased pigment therein to eradicate a majority of disease-causing germs. As discussed above, the cyclobutane dimers produced from UVC light 20 preferably cause low fidelity translation of the DNA in the disease-causing germs and leave a small population of disease-causing germ mutants that are avirulent and susceptible to antibiotics, disclosed herein as avirulent phenotype survivors susceptible to microbicides. To increase the production of avirulent phenotype survivors susceptible to microbicides, processor 30 coupled to one or more light sources 12 cycles or repeats steps a) and b) a selected number of times, e.g., two cycles shown in Table 4 below:

TABLE 4
Cycles of steps a) and b)

| Step | LED | Duty Cycle | Frequency | mW | Run Time (s) |
|---|---|---|---|---|---|
| A | UV-A | DC | | 60 | 8.333 |
| B | UV-A | 75% Duty Cycle | 10 ms | 290 | 53.70 |
| | UV-C | 80% Duty Cycle | 100 ms | 1.7 | 52.46 |
| A | UV-A | DC | | 90 | 12.50 |
| B | UV-A | 75% Duty Cycle | 100 ms | 80 | 14.81 |
| | UV-C | 60% Duty Cycle | 100 ms | 0.65 | 26.74 |

The avirulent phenotype survivors susceptible to microbicides are virtually complete depigmented making them more susceptible to clearance by the host's immune defense system. Thus, system 10 and the method thereof discussed below creates avirulent phenotype survivors susceptible to microbicides and provides a solution to the problems associated with microbicides resistance discussed in the Background section above.

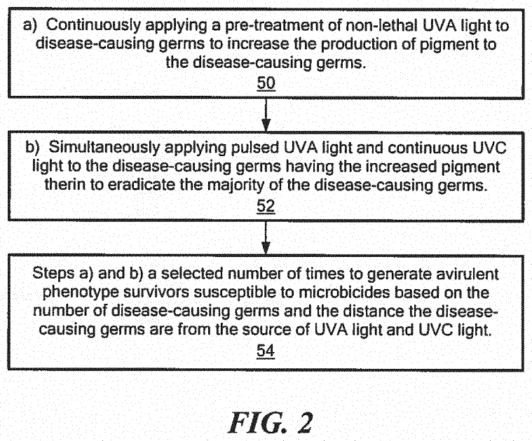

One example of the method to eradicate the majority of disease-causing germs and produce a avirulent phenotype survivors susceptible to microbicides using a light dose regiment includes: step a) continuously applying a pre-treatment of non-lethal UVB, light to disease-causing germs to increase the production of pigments in the disease-causing germs, indicated at 50, FIG. 2. Step a) selects for non-pigment expressing avirulent bacteria from within the microbial population. The method also includes step b), simultaneously applying pulsed UVA light to the disease-causing germs resulting in an increased pigment therein to eradicate the majority of disease-causing germs, indicated at 52. Step b) is lethal by converting pigments to generate ROS and also by creating cyclobutane dimers. The method also includes step c), cycling step a); and b) a selected number of times to generate avirulent phenotype survivors susceptible to microbicides, indicated at 54. In one example step a) and step b) are cycled one time. In other examples, step a) and step b) may be cycled two or more times, depending on the number of disease-causing germs and the distance of the disease-causing germs from the one or more light sources. In this example, step a) is performed for about 8 seconds (Cycle 1), and 12 seconds (Cycle 4) and step b) is performed for about 1 minute.

The number of times steps a) and b) are cycled, the light does regiment, and the characteristics of the light dose regiment discussed below, is preferably based on the number of disease-causing germs and the distance disease-causing germs 16 are from one or more light sources 12.

As disclosed herein, the light dose regiment includes the number of times each cycle of steps a) and b) are performed. In one example the light does regiment is about two cycles of steps a) and b). In another example the light does regiment is about three cycles of steps a) and b). In other examples, the light dose regiment (and characteristics of the light dose regiment discussed below) may be greater or less than two cycles of steps a) and b) depending on the number of disease-causing germs and their distance from one or more light sources. The order or characteristics of the wavelengths and waveforms may be varied.

The characteristics of the light dose regiment provided by one or more light sources 12 and processor 30 may include the wavelength of UVA light 14, the waveform of UVA light 14, e.g., the light power of UVA light 14, e.g., the wavelength of UVC light 20, e.g., the waveform of UVC light 20, e.g., the light power of UVC light 20, and/or the amount of time each of steps a) and b).

In one example, system 10 and the method thereof may generate UVA tight and UVC light in any of the following combinations of peak wavelengths: a) about 265 nm +/− about 10 nm and about 350 nm +/−15 nm, concurrently, b) about 265 nm +/− about 10 nm, and c) about 350 nm +/− about 15 nm, selectable by a user. The total light power generated by each of one or more light sources 12 may be adjusted by processor 30 over at least a range of about 0.25 mW, System 10 and the method thereof may also generate power beyond this range. System 10 and the method thereof may generate UVA and UVC light with the following waveform structures: a) continuous wave (CW), i.e. 0 Hz or DC, or b) arbitrary waveform with 100 us interval between points. System 10 and the method thereof may generate UVA light and UVC light in any of the following combinations of peak wavelengths: a) about 265 nm +/− about 10 nm and about 350 nm +/− about 15 nm, concurrently, b) about 265 nm +/− about 10 nm, and c) about 350 nm +/− about 15 nm, selectable by a user. The total light power generated by each of one or more light sources 12 may be adjusted by processor 30 over at least a range of about 0.25 mW. System 10 may also generate power beyond this range. System 10 and the method thereof may generate UVA and UVC light with the following waveform structures: a) Continuous wave (CW), i.e. 0 Hz or DC or b) Arbitrary waveform with 100 us interval between points.

In one example, the duty cycle of the UVA light and/or the UVC light provided by processor 30 and one or more light sources 12 may in the range of about 10% to about 90%. In one preferred example, the duty cycle of the UVA light and/or the UVC light is about 75%.

In one design, system 10, FIG. 1, preferably includes power supply controller 32, programmable power supplies 34, 36, drivers 38, 40, control logic 42, sensor interface 44, user interface 46, battery 49 and DC jack 50 which are coupled to processor 30 and one or more light source 12 as shown.

### Selection Process of Avirulent Antibiotic Susceptible Survivor:

### Methods:

Potential mutants of S. aures ATCC 33592 were selected by dose regiment in a biofilm irradiation experiment that presented with less yellow pigment than the wild type strain. Various levels of pigmentation were observed across the samples treated under different conditions, e.g., one treatment cycle of steps a) and b), two treatment cycles of steps a) and b), and three treatment cycles of steps a) and b), all potentially with a UV-A pretreatment, pulsed UV-A treatment and UVC direct current treatment, step b). Colonies on membrane filters from irradiated samples were selected and quadrant streaked to TSA plates. Using a sterile loop, a colony was picked off the membrane filter an added to 100 μL of 1× PBS in an Eppendorf tube. The samples in PBS were then quadrant streaked with sterile loops to TSA plates and incubated at 35° C. overnight. Colonies were selected from each condition used, and the amount of pigmentation of each sample was recorded. After overnight incubation, it was observed that the lack of pigmentation remained, as discussed in detail below. Thus, liquid cultures of the depigmented colonies were made in order to observe their growth over time compared to wild type S. aures ATCC 33592.

To assess if these potential mutations (e.g. the dose treatment selected survivors) were stable, colonies were picked from these quadrant streak plates following the same procedure and streaked again, to create a second-generation streak plate. Time course spectroscopy growth curves were then performed for both generations and correlated to serial dilution plates to enumerate colony fonning units at each spectroscopy timepoint.

### Time Course Procedure:

30 mL of TSB was added separately to five 50 mL centrifuge tubes, one tube serving as a media sterility control. A colony was picked from the S. aureus ATCC 33592 stock plate with a sterile loop and added to 30 mL of TSB. Three colonies from the overnight generation one and generation two streak plates of irradiated samples of various pigmentation were selected with sterile loops and added to TSB. One colony was selected from the samples irradiated three dose cycles (depigmented), one from the samples irradiated with two dose cycles (pale yellow), and one from the samples irradiated with one dose cycle (yellow). The optical density of each sample was measured at 600 nm. 2.5 mL of the liquid cultures was removed and added to cuvettes. The spectrophotometer was blanked with TSB and the OD was measured and recorded for each sample. The liquid cultures between time points were incubated with shaking at 37° C. At selected time points, the liquid cultures were removed from the shaking incubator and the OD was measured for each sample. The spectrophotometer was blanked in these instances with TSB also incubated at 37° C. This time course was repeated for the second-generation streak of the irradiated samples to assess if changes in growth persisted through the second generation.

### Inoculation Verification:

At each spectroscopy time point, serial dilutions of each liquid culture were performed in 1× PBS to determine the colony forming units per a (CPUs/mL) of each sample, 1 in 10 dilutions were made to various final dilutions and 100 μL of dilute culture was plated to TSA plates and incubated at 35° C. Colonies were counted post incubation to calculate CFUs/mL.

Table 5 below shows an example of time course/density determination of three selected survivors (generation 1) based on pigment phenotype from biofilm irradiation studies using the light dose regiment of cycling steps a) and h) by system 10, and the method thereof:

TABLE 5
Time Course Optical Density OD₆₀₀ of Three Selected Survivors
from Biofilm Irradiation Assay, Generation 1 Selection.

| | S. aureus ATCC 33592 (MRSA) & Survivor Isolates (Generation 1) | | | | | | | |
|---|---|---|---|---|---|---|---|---|
| | No Treatment | | | | | | | |
| | Control | | Three Cycle | | Two Cycle | | One Cycle | |
| | Parental Wild- | | Treatment | | Treatment | | Treatment | |
| Time | Type OD₆₀₀ | | Depigmented OD₆₀₀ | | Pale Yellow OD₆₀₀ | | Yellow OD₆₀₀ | |
| (hrs.) | OD₆₀₀ | CFUs/mL | OD₆₀₀ | CFUs/mL | OD₆₀₀ | CFUs/mL | OD₆₀₀ | CFUs/mL |
| 0 | 0.022 | 8.00E+05 | 0.014 | 2.00E+05 | 0.006 | 3.00E+05 | 0.006 | 4.00E+05 |
| 1 | 0.027 | 1.20E+06 | 0.001 | 2.00E+05 | 0.001 | 1.00E+05 | 0.001 | 1.00E+05 |
| 3 | 0.003 | 1.20E+06 | 0.002 | 2.00E+05 | 0.002 | 3.00E+05 | 0.002 | 1.00E+05 |
| 6 | 0.188 | 5.60E+07 | 0.008 | 2.00E+05 | 0.004 | 3.00E+05 | 0.094 | 4.70E+06 |
| 7 | 0.252 | 1.11E+08 | 0.012 | 2.00E+05 | 0.01 | 2.00E+05 | 0.15 | 2.00E+07 |
| 8 | 0.45 | 2.90E+08 | 0.003 | 2.00E+05 | 0.001 | 1.00E+05 | 0.328 | 1.30E+08 |
| 10 | 1.018 | 2.07E+09 | 0.002 | 7.52E+05 | 0.005 | 5.80E+05 | 0.616 | 9.40E+08 |
| 22 | 1.366 | 1.96E+09 | 0.008 | 4.40E+06 | 0.188 | 1.16E+08 | 1.141 | 2.06E+09 |
| 24 | 1.693 | 2.60E+09 | 0.361 | 6.00E+07 | 0.63 | 7.00E+08 | 1.511 | 3.00E+09 |
| 26 | 1.829 | 3.00E+09 | 0.892 | 4.20E+08 | 2.114 | 8.50E+09 | 1.645 | 3.70E+09 |
| 30 | 1.458 | 2.80E+09 | 2.336 | 9.70E+09 | 2.366 | 9.10E+09 | 1.87 | 4.70E+09 |

In this example, OD₆₀₀ equals Optical Density at 600 nanometer wavelength. Absorbance readings of generation 1 samples of avirulent phenotype survivors susceptible to microbicides at 600 nm. At various time points indicated, samples were removed from shaking incubator to read absorbance and assess cell density. Approximately 2.5 mL of each sample was added to a cuvette. The spectrophotometer was blanked with 2.5 mL of TSB (also incubated at 37° C.) and then the absorbance was read and recorded for each sample. CFUs: 1 in 10 dilutions were performed on each sample as an inoculation verification. 100 μL of various dilutions were plated to TSA plates and incubated at 35° C. and enumerated for CFUs. CFUs/mL is the average at the least dilute dilution where countable colonies were obtained multiplied by the reciprocal of the dilution and the plating factor (10×). Parental Wild-Type equals Methicillin Resistant S. aureus ATCC 33592, Treatment equals Dose Regiment, Cycle number equals the amount of times the light dose regiment of cycling step a) and step b) was applied by system 10 and the method thereof to produce the avirulent phenotype microbicides. The avirulent phenotype microbicides in this set of experiments was produced after three cycles of steps a) and b) and is characterized by a lack of pigmentation.

Table 6 below shows an example of time course optical density of three selected survivors (Generation 2) from biofllm irradiation assays:

TABLE 6
time course optical density OD₆₀₀ of three selected survivors from biofilm irradiation
assay, generation 2 of avirulent phenotype microbicides survivors selection I.

| | S. aureus ATCC 33592 (MRSA) & Survivor Isolates (Generation 2) | | | | | | | |
|---|---|---|---|---|---|---|---|---|
| | | | Three Cycle | | | | | |
| | No Treatment | | Treatment | | | | | |
| | Control | | Depigmented OD₆₀₀ | | Two Cycle | | One Cycle | |
| | Parental Wild- | | | CFUs/machine | Treatment | | Treatment | |
| Time | Type OD₆₀₀ | | | learning | Pale Yellow OD₆₀₀ | | Yellow OD₆₀₀ | |
| (hrs.) | OD₆₀₀ | CFUs/mL | OD₆₀₀ | subsystem 122 | OD₆₀₀ | CFUs/mL | OD₆₀₀ | CFUs/mL |
| 0 | 0.002 | 1.13E+05 | 0.001 | 3.27E+05 | 0.001 | 3.83E+05 | 0.002 | 4.50E+05 |
| 1 | 0.005 | 1.79E+05 | 0.003 | 1.00E+06 | 0.004 | 3.00E+05 | 0.002 | 8.10E+05 |
| 3 | 0.005 | 2.02E+05 | 0.002 | 1.95E+06 | 0.001 | 8.40E+05 | 0.005 | 2.18E+06 |
| 6 | 0.019 | 2.64E+06 | 0.012 | 8.60E+05 | 0.131 | 5.40E+07 | 0.272 | 1.07E+08 |
| 7 | 0.052 | 8.00E+06 | 0.006 | 4.90E+05 | 0.386 | 1.90E+08 | 0.573 | 5.70E+08 |
| 8 | 0.116 | 5.60E+07 | 0.006 | 3.10E+05 | 0.589 | 7.10E+08 | 0.837 | 1.21E+09 |
| 10 | 0.668 | 1.00E+09 | 0.004 | 1.10E+05 | 1.186 | 2.18E+09 | 0.612 | 2.39E+09 |
| 22 | 1.712 | 2.43E+09 | 0.862 | 2.50E+08 | 1.596 | 3.30E+09 | 1.567 | 2.84E+09 |
| 24 | 1.693 | 3.20E+09 | 1.601 | 4.50E+09 | 1.618 | 2.40E+09 | 1.559 | 2.90E+09 |
| 26 | 1.719 | 2.70E+09 | 1.848 | 4.00E+09 | 1.641 | 5.10E+09 | 1.59 | 3.00E+09 |
| 30 | 1.738 | 4.50E+09 | 1.913 | 2.40E+09 | 1.665 | 3.20E+09 | 1.598 | 2.70E+09 |

In this example, OD₆₀₀ equals Optical Density at 600 nanometer wavelength, Absorbance readings of generation 1 samples at 600 nm. At various time points indicated, samples were removed from shaking incubator to read absorbance and assess cell density. Approximately 2.5 mL of each sample was added to a cuvette. The spectrophotometer was blanked with 2.5 mL of TSB (also incubated at 37° C.) and then the absorbance was read and recorded for each sample.

In this example, CFUs: 1 in 10 dilutions were performed on each sample as an inoculation verification. 100 μL of various dilutions were plated to TSA plates and incubated at 35° C. and enumerated for CPUs. CRUs/mL is the average at the least dilute dilution where countable colonies were obtained multiplied by the reciprocal of the dilution and the plating factor (10×) Parental Wild-Type equals Methicillin Resistant S. aureus ATCC 33592, Treatment equals Dose Regiment, Cycle number equals the amount of times the light dose regiment of step a) and step b) were applied to produce the avirulent phenotype microbicides. The avirulent phenotype microbicides in this set of experiments was produced after three cycles and is characterized by a lack of pigmentation.

### Power Example (1):

Step a): 1 UV-A Proximal Power: 34.64 mW @ 4.5V DC, 60 seconds. Step b): 1 UV-A Proximal Power: 25.98 mW @ 4.5V Pulse, 100 Hz, 75% Duty Cycle, 1200 seconds UV-C Proximal Power: 0.246 mW @ 9V DC, 1200 second.

### Power Example (2):

Step a): 2 UV-A Proximal Power: 34.64 mW @ 4.5V DC. 120 seconds, Step b): 2 UV-A Proximal Power: 25.98 mW @ 4.5V Pulse, 100 Hz, 75% Duty Cycle, 480 seconds UV-C Proximal Power: 0.246 mW @ 9V DC, 480 seconds.

### Power Example (3):

Step a): 3 UV-A Proximal Power: 34.64 mW @ 4.5V DC, 60 seconds. Step b): 3 UV-A Proximal Power: 25.98 mW 4.5V Pulse, 100 Hz, 75% Duty Cycle, 180 seconds UV-C Proximal Power: 0.246 mW @ 9V DC, 180 seconds.

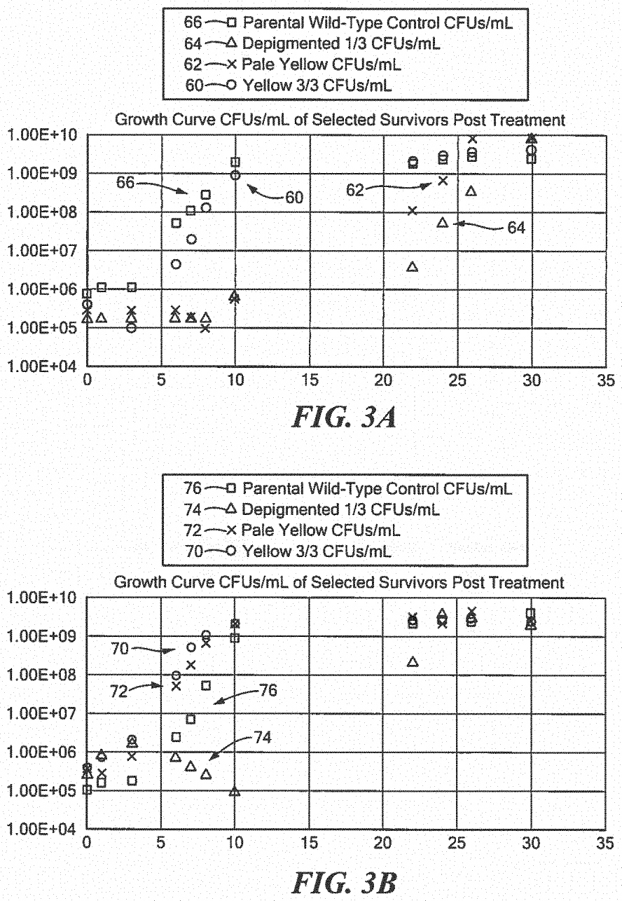

Growth curve 60, FIG. 3A, for Table 5 above, shows an actual growth curve for the wild type S. aureus ATCC33592 (fully virulent and fully pigmented). Growth curve 62 shows an example of the growth curve for S. aureus after a minimal exposure to a light dose regiment of steps a) and b) discussed above. In this example, the pigment in the S. aureus is reduced to a pale yellow color. Growth curve 64 shows an example of the growth curve for S. aureus after a light dose regiment of step c) cycling steps a) and b) two times which produces the avirulent phenotype survivors susceptible to microbial agents. The S. aureus is completely depigmented and therefore are susceptible to microbial agents. Growth curve 66 is the parental wild-type control.

Growth curve 70, FIG. 3B, for Table 6 above, shows an actual growth curve for the wild type S. aureus ATCC33592 (fully virulent and fully pigmented). Growth curve 72 shows an example of the growth curve for S. aureus after a minimal exposure to a light dose regiment of steps a) and b) discussed above. In this example, the pigment in the S. aureus is reduced to a pale yellow color. Growth curve 4 shows an example of the growth curve for S. aureus after a light dose regiment of step c) cycling steps a) and b) two times which produces the avirulent phenotype survivors susceptible to microbial agents. The S. aureus is completely depigmented and therefore are susceptible to microbial agents. Growth curve 76 is the parental wild-type control.

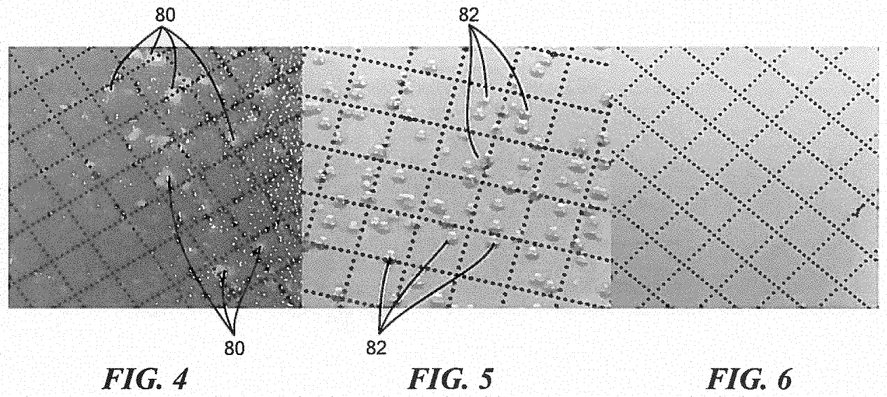

FIG. 4 shows one example a control growth of S. aureus before exposure to cycling steps a) and b) a selected number of times using the light dose regiment of UVA light and UVC light discussed above. The fully pigmented disease-causing germs, in this example, S. aureus are exemplarily indicated at 80.

FIG. 5 shows an example of the S. aureus shown in FIG. 4 after cycling steps a) and b) one time using a light dose regiment of Power Example (2) above for about 8 minutes using UVA light at light power of about 1256.4 mJ and UVC light at a light power of about 13.5 mJ directed several cm away from the light source to a biofilm of 5 million disease-causing cells, exemplarily indicated at 82. As can be seen in FIG. 5. the pigmentation of the disease-causing germs, in this example, S. aureus, is significantly reduced. In this example, log cells of S. aureus killed or eradicated was 6.74 and CFUs survivors was 400.

FIG. 6 shows an example disease-causing germs, in this example, of the S. aureus shown in FIG. 4, after a light dose regiment of Power Example 3 cycled one time by system 10 and the method thereof indicated at 54 using UVA light at light power of about 3141 mJ and UVC light at a light power of 33.7. As can be seen in FIG. 6, the S. aureus is completely depigmented resulting in the production of aviculent phenotype survivors susceptible to microbial agents. In this example, log cells of S. aureus killed or eradicated was 6.74 and CFUs survivors was 1.

Table 7 below show and example of a Clinical laboratories Science Institute (CLSI) reference table typically used by clinicians and clinical microbiology laboratories to establish whether a clinical isolate is susceptible, intermediate or resistant to a particular type of antibiotic under the Kirby-Bauer ZOI assay:

TABLE 7
CLSI for Antimicrobial Agents, Standard Zone Diameters
Zone diameter (mm)

| Antimicrobial agent | Susceptible | Intermediate | Resistant |
|---|---|---|---|
| Linezolid | >21 | — | ≤20 |
| Norfloxacin | * | * | * |
| Ofloxacin** | ≥18 | 15-17 | ≤14 |
| Ciprofloxacin** | ≥21 | 16-20 | ≤15 |
| Cefprozil | ≥22 | — | ≤21 |
| Rifampin | >20 | 17-19 | ≤16 |
| Oxacillin | * | * | * |
| Tetracycline | >19 | 15-18 | ≤14 |

In one example, the Kirby-bauer Zones of Inhibition (Z01) assay was performed on the untreated MRSA parent strain and the two generations of the depigmented avirulent phenotype survivors susceptible to microbicides were created by applying the light dose regiment of system 10 and the method thereof discussed above based on the CLSI guidelines, See Table 8 below:

TABLE 8
Antibiotic Susceptibility Testing, Survivor, Depigmented Zone
(diameters in mm, R = resistant, S = susceptible)

| | S. aureus | Depigmented | Depigmented |
|---|---|---|---|
| Antibiotic | ATCC 33592 (MRSA) | Generation 1 | Generation 2 |
| Linezolid | 31.33 (S) | 35.11 (S) | 32.8 (S) |
| Norfloxacin | 27.67 (S) | 32.68 (S) | 30.17 (S) |
| Cefprozil | No Inhibition (R) | 42.99 (S) | 42.98 (S) |
| Rifampin | No Inhibition (R) | 45.04 (S) | 48.68 (S) |
| Oxacillin | No Inhibition (R) | 28.04 (S) | 27.25 (S) |
| Tetracycline | No Inhibnion (R) | 34.66 (S) | 31.31 (S) |

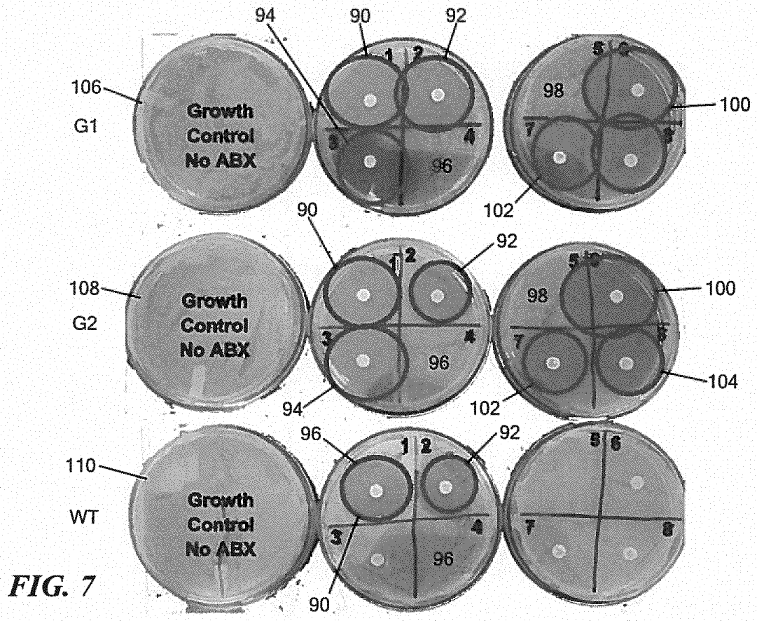

FIG. 7 shows examples of Zones of Inhibition (ZOI) for the Kirby-bauer ZOI assay performed above which determines the lowest concentration of an antimicrobial agent that prevents visible growth of a microorganism for two consecutive generations of an avirulent selected MRSA phenotype. The first generation (G1) is the avirulent phenotype survivors susceptible to microbicides created by system 10 and the method thereof and the second generation (G2) is the avirulent phenotype survivors susceptible to microbicides created by system 10 and the method thereof. In this example, the ZOI for Linezolid is indicated at 90, the ZOI for Norfloxacin is indicated at 92, the ZOI for Cefprozil is indicated at 94, ZOEs 96 and 98 are blank, ZOI 100 is for Rifampin, ZIO 102 is for Oxacillin and ZOI 104 is for Tetracycline. The growth control for Generation 1 (G1), Generation 2 (G2) and the wild type (WT) is indicated at 106, 108 and 110, respectively. In this example, Methicillin Resistant, S. aureus ATCC 33592. In this example, WT is Oxacillin is considered equivalent to methicillin when describing a MRSA strain. In this example, the ZOIs were measured at 24 hours and 72-hours past-challenge. Generation 1 was serially passaged onto an antibiotic-free TSA plate, then transferred to TSB liquid culture prior to performing the ZOI assay. Generation 2 was transferred from Generation 1, serially passaged onto an antibiotic-free TSA plate, then transferred to a TSB liquid culture prior to performing the ZOI assay. The generations demonstrate that the drug susceptible phenotype is not transient. Thus, the avirulent phenotype survivors susceptible to microbicides are stable over multiple generations.

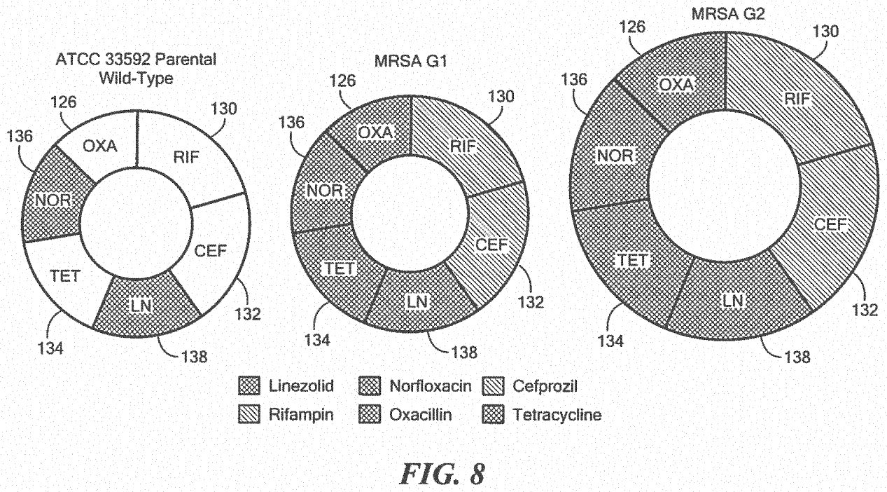

FIG. 8 shows a heatmap profile showing an example of the resistance and susceptibility of S. aureus ATCC 33592 Parental Wild-Type to various antibiotics, indicated by panel 120, compared to depigmented Generation 1 of avirulent phenotype survivors susceptible to microbicides (MRSA G1), in indicated by panel 122, and compared to Generation 2 (MRSA G2) of the depigmented avirulent phenotype survivors susceptible to microbicides, in this example as specified by CLSI MI00-ED29:2019, indicated by panel 124. See e.g., Clinical and Laboratory Standards Institute, Performance Standards for Antimicrobial Susceptibility Testing, 29th Edition 950 West Valley Road Suite 2500, Wayne, Pa. 19087 USA, in this example Oxacillin is synonymous with methicillin when describing MRSA.

In this example, panel 120 shows the S. aureus ATCC 33592 Parental Wild-Type is resistant to Oxacillin, indicated at 126, Rifampin, indicated at 130, Cefprozil, indicated at 132, and Tetracycline, indicated at 134 and susceptible Norfloxacin, indicated at 136, and Linezolid, indicated at 136. After treatment with the light dose regiment of cycling steps a) and b) of system 10 and the method thereof discussed above, panel 122 for MRSA G1 (Generation 1) and panel 124 for MRSA G2 (Generation 2) show Oxacillin, indicated at 126, Rifampin, indicated at 130. Cefprozil, indicated at 132, and Tetracycline, indicated at 134, are now susceptible to microbicides.

The result shows that for four antibiotics, Cefprozil, Rifampin, Oxacillin (e.g, Methicillin), and Tetracycline, the drug resistant strain, the avirulent phenotype survivors susceptible to microbicides, are susceptible to microbicides after treatment to the light dose-regiment treatment of system 10 and the method thereof discussed above. Thus, the light dose regiment of system 10 and the method thereof clearly converts Methicillin-resistant Staphylococcus aureus into a mutagenic-stable drug-susceptible variant.

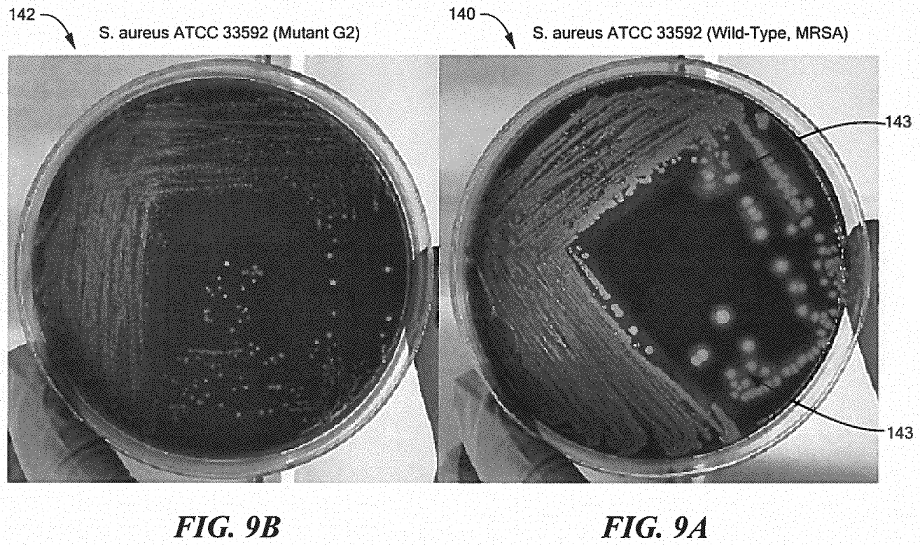

In another example, a quadrant streak was performed onto sheep blood agar (TSA w/5% sheep blood) to compare the MRSA wild-type strain to the Generation 2 (G2) (antibiotic susceptible stable mutant) to determine whether the antibiotic susceptible phenotype was also unable to metabolize hemoglobin. FIGS. 9A and 9B show the results of the test. The Parental Wild-Type MRSA strain (ATCC 33592) is indicated at 140 after 48 hours on sheep blood agar (TSA with 5% sheep blood) compared to depigmented avirulent phenotype survivors susceptible to microbicides, in this example mutant Generation 2, indicated at 142. The halo around the wild-type exemplarily indicated at 143, FIG. 9A, represents lysis of red blood cells by hemolysin, a protein that destroys the membrane of red blood cells. Hemolysins are virulence factors. The depigmented avirulent phenotype survivors susceptible to microbicides, mutant Generation 2, indicated at 142, reveals no zone of lysis.

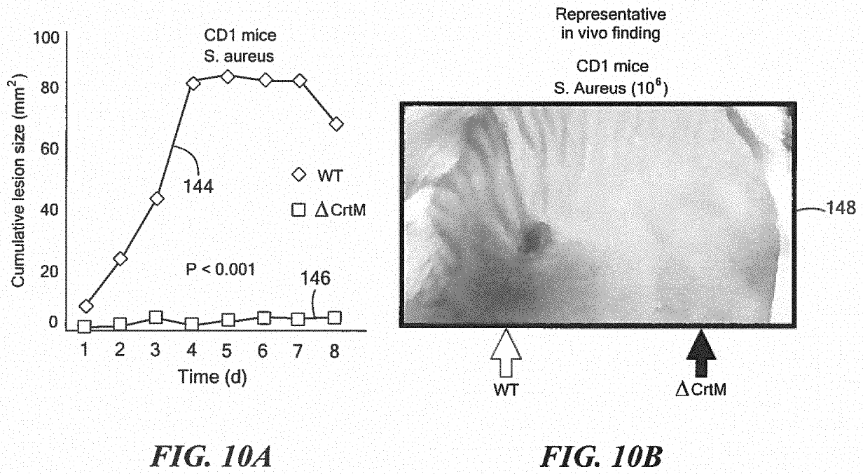

FIG. 10A shows an example for the contribution of the 5% S. aureus staphyloxanthin (carotenoid pigment) to virulence in vivo by direct comparison of an isogenic pair of a wild type (WT) with a knock out rionpigmented mutant strain (ΔCrtM). A subcutaneous abscess mouse model is used. Graphs 144, 146 represent the cumulative lesion size generated by the respective bacterial strain in mm². Image 148, FIG. 10B, shows representative mice from the corresponding challenge groups (WT versus ΔCrtM). See, e.g., George Y. Liu, Anthony Essex, John T, Buchanan, Vivekariand Datta, Hal M. Hoffman, John F, Bastian, Joshua Fierer, Victor Nizet; Staphylococcus aureus golden pigment impairs neutrophil killing and promotes virulence through its antioxidant activity. J Exp Med 18 Jul. 2005; 202 (2): 209-215. doi: http//doi.org/10.1084/jem.20050846, incorporated by reference herein.

In another example, results from the absorption spectra for methanol extracted Staphyloxanthin are consistent with results reported elsewhere in the literature for wild-type S. aureus. Staphyloxanthin is a carotenoid pigment produced by some strains of Staphylococci and is the pigment responsible for the “golden color”. It is an antioxidant protects the microorganism from reactive oxygen species (ROS) such as peroxides produced by the host defense system (macrophages). ROS production also occurs during UV irradiation at both about 365 nm and at about 265 nm. Thus, pigmentation can be considered to confer protection against UV photodamage. The pigment is required for virulence and studies by Liu et al. cited supra have shown that genome deletion of crtM, the principle bio-synthetic pathway necessary to produce Staphyloxanthin are unable to produce lesions when injected into the back of mice. In one example, selected mutants, e.g., depigmented survivor Generation 1 and Generation 2, the avirulent phenotype survivors susceptible to microbicides created by the light dose regiment of system 10 and the method thereof, produced quantities of pigment at least six times less to the parental strain (Methicillin Resistant S. aureus ATCC 33592). If fact, the strains of avirulent phenotype survivors susceptible to microbicides may in fact not produce any pigment, as 0.2 is the limit of detection for the spectrophotometric assay. See e.g., Liu G. et al., Staphylococcus aureus Golden Pigment Impairs Neutrophil Killing and Promotes Virulence Through its Antioxidant Activity J Exp Med, 202:209-15 (2005) and Dong P. et al Photolysis of Staphyloxanthin in Methicillin-Resistant Staphylococcus aureus Potentiates Killing by Reactive Oxygen Species, Adv Sci (Weinh), 6:1900030 (2009), both incorporated by reference herein.

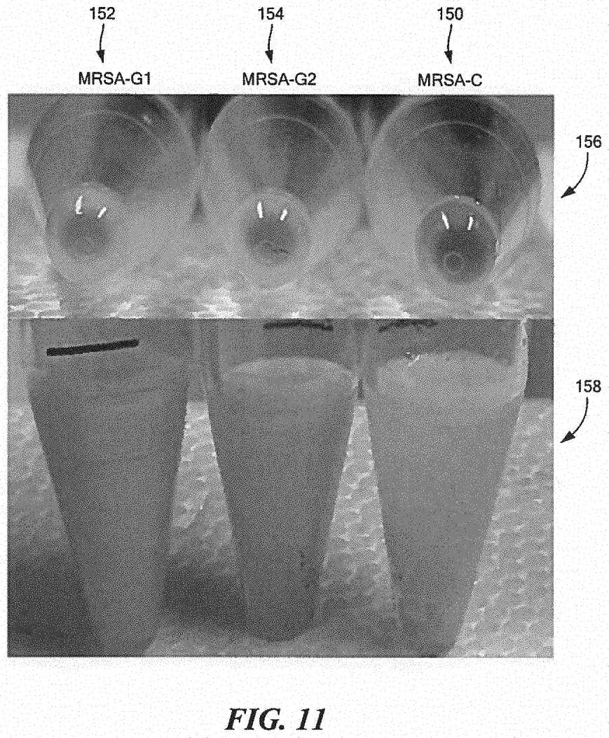

FIG. 11 shows an example of MRSA-C, Methicillin Resistant S. aureus ATCC 33592, indicated at 150, depigmented survivor first generation, MRSA-G1, avirulent phenotype survivors susceptible to microbicides, indicated at 152, and depigmented survivor second generation, MRSA-G2, avirulent phenotype survivors susceptible to microbicides, indicated at 154. Top panel 156 shows an example of centrifuged pellets of the whole cell fraction and bottom panel 158 shows an example of Staphyloxanthin pigment following methanol extraction.

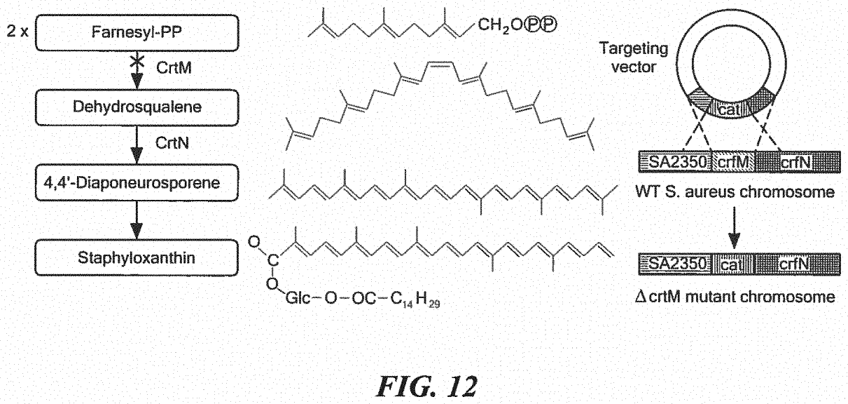

FIG. 12 shows an example of the genetic deletion in the carotenoid pigment pathway to eliminate pigment production in S. aureus. See e.g., Liu G. et al., Staphylococcus aureus Golden Pigment Impairs Neutrophil Killing and Promotes Virulence Through its Antioxidant Activity, cited supra, FIG. 10B discussed above also shows the deleted pigment (delta crtM) is the strain possessing the disrupted pathway for pigmentation.

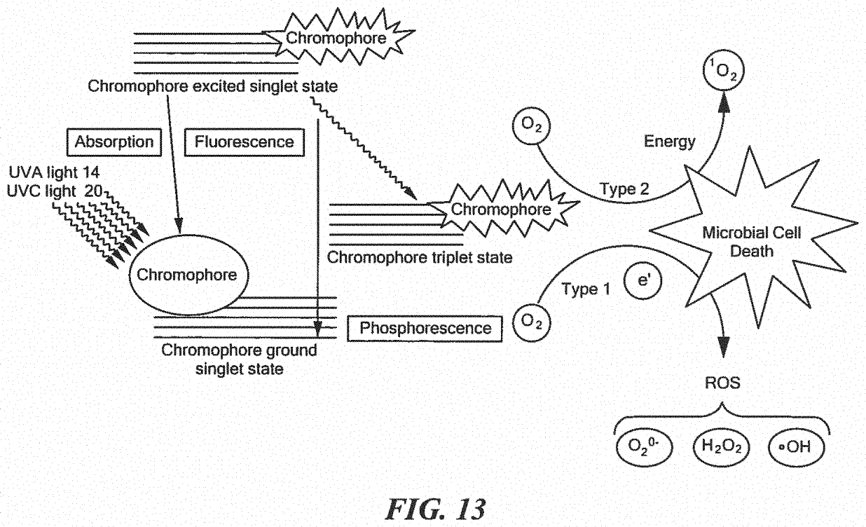

FIG. 13 shows a schematic illustration of virulence photoinactivation, including the Jablonski diagram provided by the application of UVA light and UVC light dose regiment by system 10 and the method thereof discussed above. Endogenous photoactive chromophores (including pigments) excitation to the first singlet state initiated by photon absorption can relax to the longer-lived triplet state. Triplet PS interacts with molecular oxygen in type I and type II pathways leading to ROS and ₁O² formation, respectively, and death of disease-causing germs.

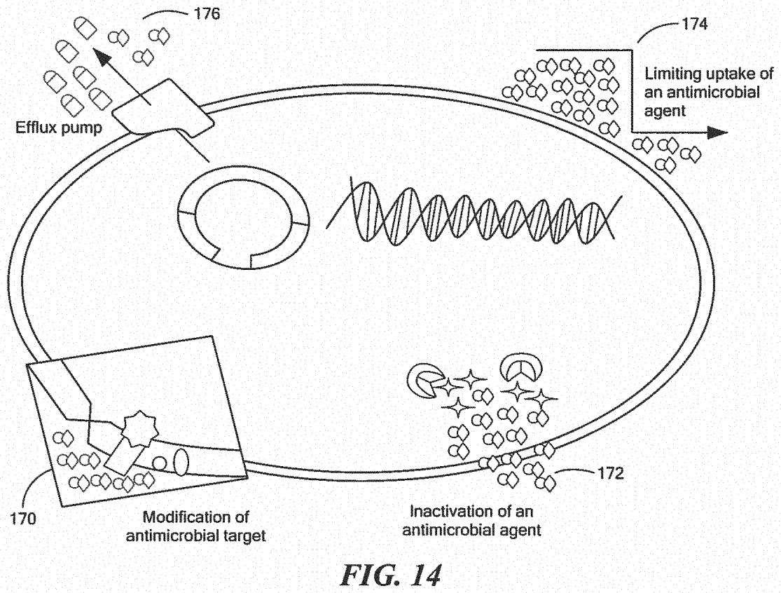

FIG. 14 shows an example of four mechanisms utilized by disease-causing germs to prevent their susceptibility to microbicides. These include modification of microbicides, indicated at 170, inactivation of the microbicides indicated at 172, limiting uptake of the antimicrobial agent indicated at 174, and efflux pump indicated at 176. Intrinsic efflux mechanisms are broadly recognized as major components of resistance to many classes of antimicrobials. Efflux occurs due to the activity of membrane transporter proteins, widely known as Multidrug Efflux Systems. Identification of natural substrates and Efflux Pump Inhibitors (EPIs) is an active research discipline and many structurally and functionally diverse compounds have been identified. A brief survey of the efflux systems in pathogens illustrates the biological complexity, overlapping substrate specificity and the evolutionary adaptability to antimicrobials. Multidrug resistance in Gram-positive bacteria is attributed to members of the chromosomally encoded Major Facilitator Superfamily (MFS, NorA, NorB, NorC, MdeA), the MATE mepRAB (multidrug export protein) and the SMR SepA. There is also evidence of a key role for plasmid encoded systems, such as QacA, QacB, and Tet(K), which function as tetracycline-divalent metal complex/H⁺ antiporters. The most clinically significant efflux systems in Gram-negative bacteria are the Resistance Nodulation and cell Division (RND) tripartite transporters, which show broad substrate recognition profiles, The RND family comprises proton-driven systems, such as the E. coli AcrAB-TolC; AcrB functions as a multi-subunit complex in association with the outer membrane channel TolC, and the membrane fusion protein AcrA. Although the crystal structure has been resolved and the pump components re-assembled by modelling, the exact role of each efflux component remains elusive. P. aeruginosa carries 12 related Mex efflux systems of which four (MexAB-OprM, MexCD-OprJ, MexEF-OprN, MexXY-OprM) have been shown to be clinically important for intrinsic resistance. At least five ade-RND systems are functionally characterized in A. baumannii, including AdeIJK, adeSR-adeABC, adeDE and AdeFGH. These systems show broad, overlapping substrate specificity for quinolones, tetracyclines, monovalent and divalent antimicrobial cations (intercalating dyes, quaternary ammonium compounds, diamidines, biguanidines, bile salts) and many plant secondary metabolites. See, e.g., Ball et al., Microbial efflux systems and inhibitors: approaches to drag discovery and the challenge of clinical implementation, Open Microbial J., 3;7:34-52 (2013), incorporated by reference herein.

The light dose regiment provided by system 10 and the method thereof discussed above with reference to one or more of FIGS. 1-14 effectively produces avirulent phenotype survivors which cannot utilize the four mechanisms discussed above to prevent their susceptibility to microbicides to produce the avirulent phenotype survivors susceptible to microbicides. The light dose regiment provided by system 10 and the method thereof discussed above also preferably interferes with gene regulation, transcription, and translation yielding an avirulent survivors phenotype that is characterized by a permanently and genetically altered state unable to maintain normal physiological gene regulation, including production of virulence factors, and germicidal resistance mechanisms to microbicides thereby rendering the avirulent phenotype survivors susceptibility to microbicides and non-infectious.

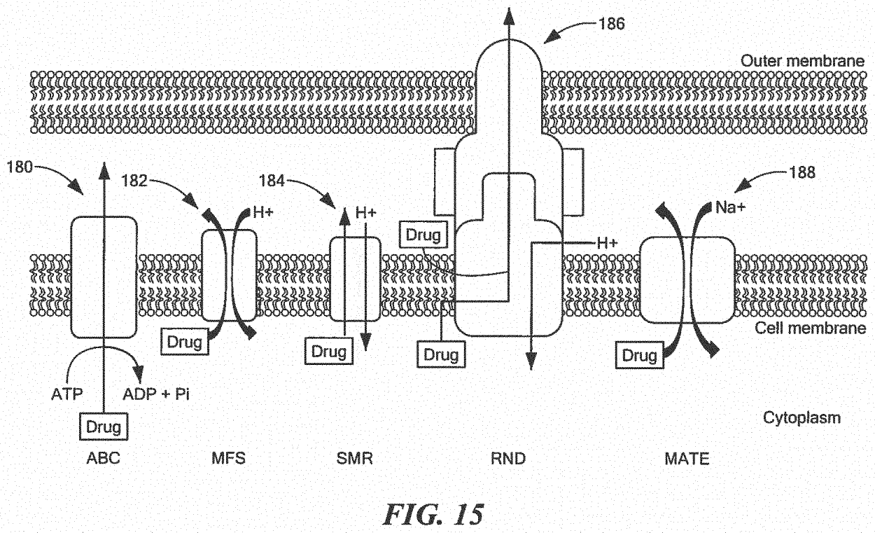

FIG. 15 shows a schematic illustration for examples of key members of the five super-families of the microbial efflux system 176 shown in FIG. 14: ATP-binding cassettes (ABC), indicated at 180, FIG. 15, major facilitators (MFS), indicated at 182, small multidrug resistance family (SMR), indicated at 184, resistance nodulation cell division (RND), indicated at 186, and multi-antimicrobial extrusion protein family (MATE), indicated at 188. See e.g., Du, D. et al Multidrug Efflux Pumps: Structure, Function and Regulation, Nat. Rev. Microbial., 16, 523-539 (2018), incorporated by reference herein.

As discussed in the above, persister cells are a continually reoccuring sub-population that when located in a biofilm can survive antibiotics by going into a state of dormancy and that such a process is produced through a highly regulated and sophisticated regulatory mechanism of gene expression.

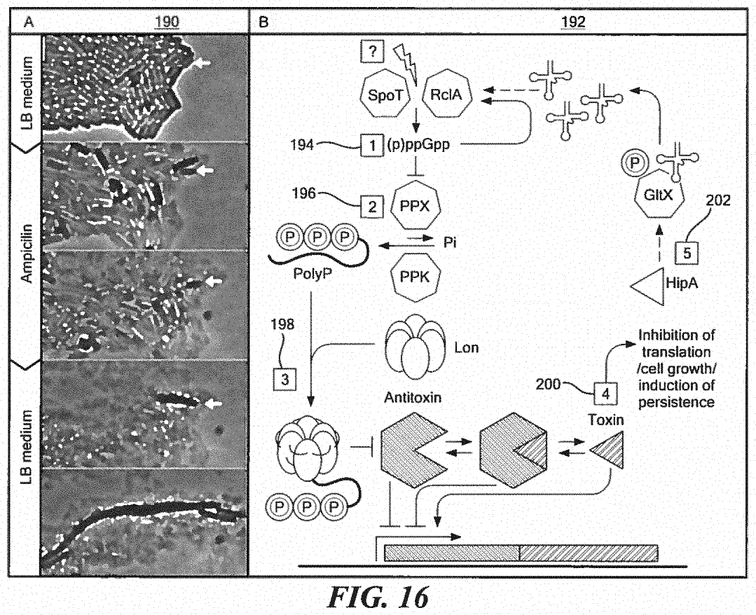

Illustrative Model A, indicated at 190, FIG. 16, shows an example of a time-lapse microscopy experiment showing that single E. coli cells with a high level of [ppGpp] as revealed by expression of the “ppGpp-proxy” RpoS-mCherry survive antibiotic treatment, Model B, indicated at 192, shows a model example for how ppGpp stochastically induce persistence in E. coli. Activation of RelA or SpoT in single cells promotes accumulation of ppGpp is indicated at 1-194. The ppGpp-dependent inhibition of exopolyphosphatase (PPX), indicated at 2-196, shows the cellular enzyme that degrades PolyP, promotes PolyP accumulation by the constitutively active polyphosphate kinase (PPK). PolyP combines with and stimulates Lon to degrade all 11 type II antitoxins of E. coli K-12, indicated at 3-198. Free and activated toxins inhibit translation and cell growth and thereby induce persistence, indicated at 4-200. Speculative positive-feedback loop that ensures even more synthesis of ppGpp, indicated at 5-202. The above model predicts that degradation of HipB enables free HipA to phosphorylate glutamyl-tRNA synthetase (GltX) and inhibits charging of tRNAGlu. The resulting uncharged tRNAs enter the ribosomal A site and trigger RelA-dependent synthesis of (p)ppGpp. See e.g., Maisonneuve et al, (2014), incorporated by reference herein. Maisonneuve E, Gerdes K. Molecular mechanisms underlying bacterial per inters. Cell. 2014;157(3):539-548. doi:10.10161/j.cell.2014.02.050.

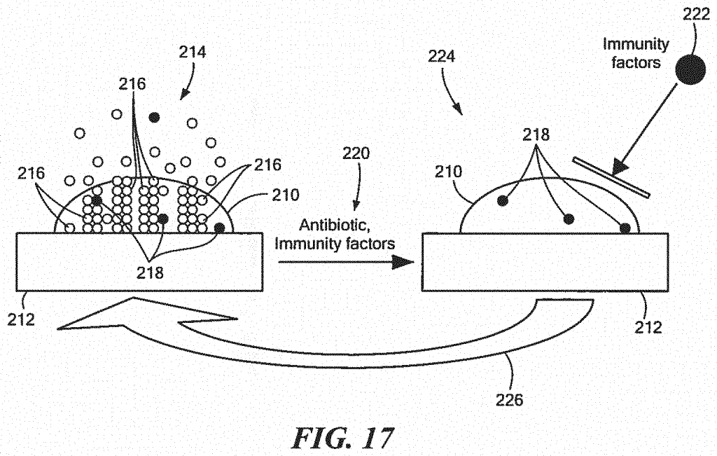

FIG. 17 shows an example model of biofilm resitance based on persister survial discussed above. In this model, biofilm 210 on substrate 212 on the left, indicated at 214, houses planktonic persisters generally indicated at 216 and biofilm persisters indicated at 218. An initial treatment with microbicides, such as antibiotic, indicated at 220, kills the planktonic cells and the majority of biofilm cells. The immune system, e.g., immuity factors 222, kills planktonic persisters 216 but biofilm persisters 218 are protected from host defenses by the exopolysaecharide matrix of biofilm 210, indicated on right at 224. After the concentration of the microbicide drops, e.g., an antibiotic, indicated at 226, planktonic persisters 216 resurrect in the biofilm, indicated at 214, and the infection reoccures.

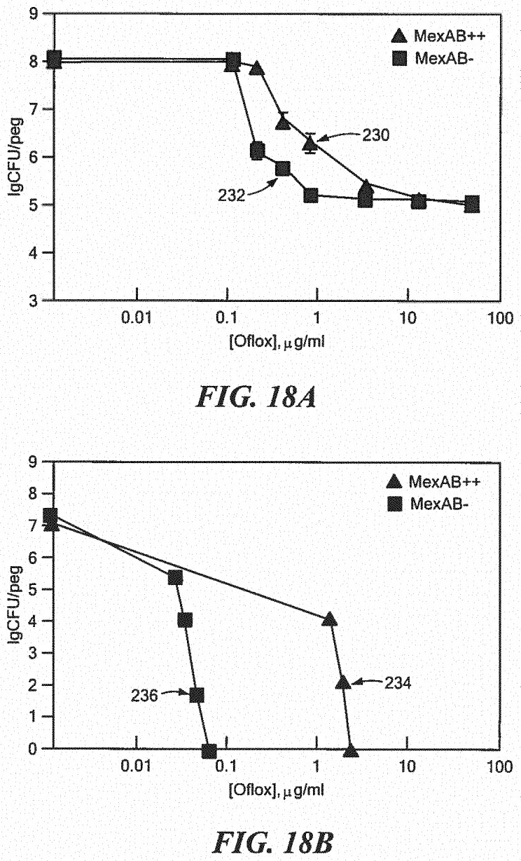

FIG. 18A, where a microbial biofilin is present, shows examples of P. aeruginosa persisters surviving in a biofilm treated with ofloxacin (Oflox) in this example, biofilms were formed on pegs of a Calgary Biofilm Device and were then treated with a given concentration of antibiotic in Mueller-Hinton broth for 6 h, rinsed, and dislodged by sonication. Live cells were then counted by plating. The number of live cells recovered from a single peg is expressed as the number of CFU per peg. A strain that overexpressed the main MDR pump that extrudes fluoroquinolones, MexAB⁺⁺, indicated by curve 230 and a strain that lacked the efflux pump, MexAB′, is indicated by curve 232, were used in this experiment. The contribution of the efflux pump to resistance is evident at low concentrations of the antibiotic but has little effect on the survival of persisters. Curve 234, FIG. 18B, where no microbial biofilm is present, for MexAB⁺⁺ and curve 236, for MexAB⁺, shows examples where planktonic cells were treated similarly with ofloxacin and plated for determination of the cell count. The apparent absence of persisters shown by curves 234 and 236 is due to the low density of the population and the detection limit of the experiment. At higher densities, persisters are evident at low levels in a planktonic population. See e.g., Lewis K., Riddle of Biofilm Resistance cited supra. Appling the light dose regiment of cycling steps a) and b) the selected number of times by system 10 and the method thereof discussed above generates avirulent phenotype survivors susceptible to microbicides which are unable to produce a microbial biofilm associated with reoccurring infections. Appling the light dose regiment of cycling steps a) and b) the selected number of times also converts persisters in a microbial biofilm associated with reoccurring infections from a dormant state to a non-dormant state thereby making the persisters susceptible to microbicides.

### Transcriptomics:

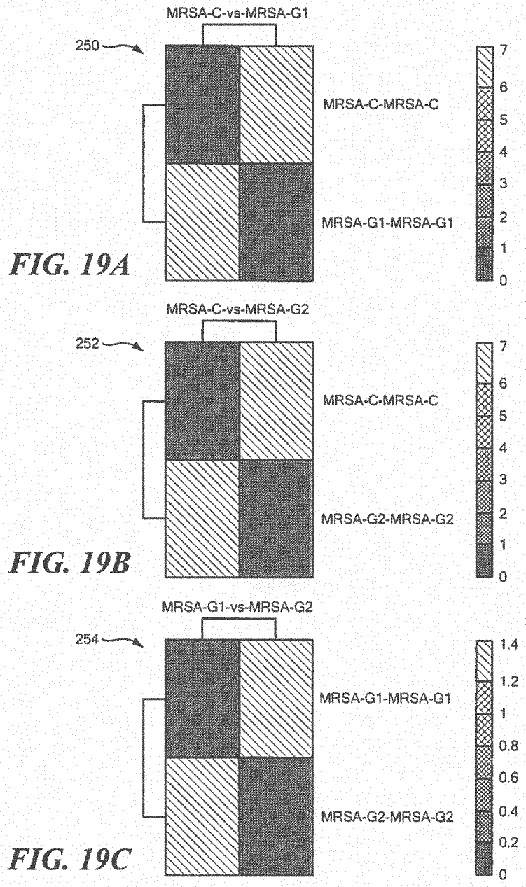

Plot 250, FIG. 19A, shows an example of sample distance, S. aureus ATCC 33592 Parental Wild-Type (MRSA-C) to Depigmented Generation 1 avirulent phenotype survivors (MRSA-G1) generated by system 10 and the method thereof discussed above. Plot 252, FIG. 19B, shows an example of sample distance, S. aureus ATCC 33592 Parental Wild-Type (MRSA-C) to Depigmented Generation 2 avirulent phenotype survivors (MRSA-G2) generated by system 10 and the method thereof discussed above. Plot 254, FIG. 19C, shows an example of sample distance, Depigmented Generation 1 avirulent phenotype survivors (MRSA-G1) generated by system 10 and the method thereof discussed above to Depigmented Generation 2 avirulent phenotype survivors (MRSA-G2) generated by system 10 and the method thereof discussed above.

FIGS. 19A, 19B, and 19C show the sample distances measured using expression values from each sample, The shorter the distance, the more closely related the samples are. This method is used to identify if the two groups are closely related or not. The heatmap of sample-to-sample distance matrix provides an overview over the similarities and dissimilarities between samples: in the all three comparisons in plots 250, 252, and 254, the samples appear distinct and dissimilar. The scale is in Log₁₀. This demonstrates that the profile of transcription is completely dissimilar to the parental control (MRSA-C) for both MRSA-G1 and MRSA-G2.

Each shade of difference, indicated by shade grid 256 for FIG. 19A, shade grid 258 for FIG. 19B, and shade grid 260 for FIG. 19C, indicates s a difference of 100%. Therefore MRSA-G1 and MRSA-G2 are both 700% different in transcription than the parental strain MRSA-G1. Importantly, each shade of difference also shows that the MRSA-G1 and MRSA-G2, are isotypes of each other (e.g. identical). Furthermore, this kind of distance between similar species is not expected. This indicates that cellular regulation is uncontrolled. Since virulence, persistence, and antibiotic resistance all require sophisticated cellular regulation, this transcriptomics data supports the premise that the avirulent antibiotic phenotype is due to art ability to control regulation.

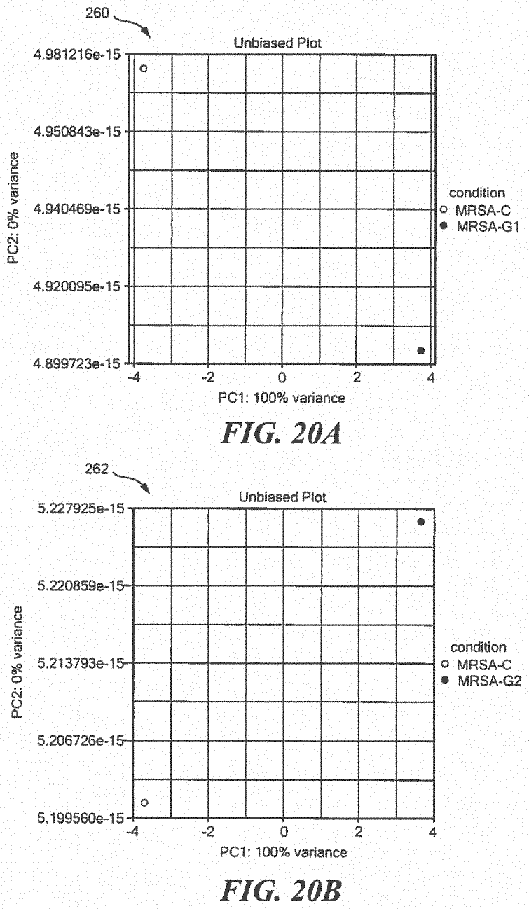

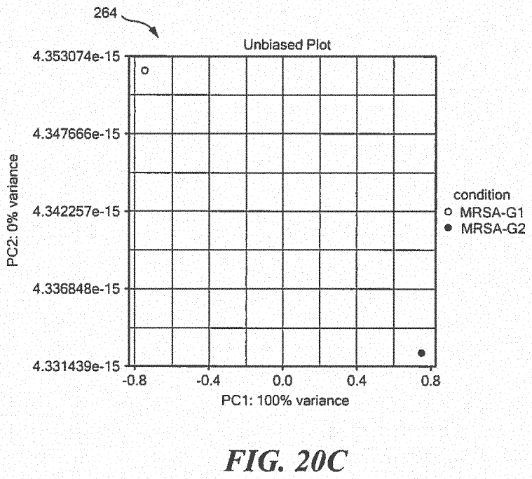

Plot 260, FIG. 20A, shows an example of principal component analysis (PCA) of S. aureus ATCC 33592 Parental Wild-Type (MRSA-C) to Depigmented Generation 1 avirulent phenotype survivors (MRSA-G1), Plot 262, FIG. 20B, shows an example of PCA of S. aureus ATCC 33592 Parental Wild-Type (MRSA-C) to Depigmented Generation 2 avirulent phenotype survivors (MRSA-G2). Plot 264, FIG. 20C, shows an example of PCA of Depigmented Generation 1 avirulent phenotype survivors (MRSA-G1) to Depigmented Generation 2 avirulent phenotype survivors (MRSA-G2).

The PCA analysis in FIGS. 20A, 20B, and 20C illustrates the separation between the three phenotypes and replicates the conditions tested. Experimental variance was not determined with the number of replicates tested therefore experimental covariates and batch effects were not determined. This is an exploratory analysis to reveal the similarity within and between groups. The groups are plotted based on their closeness and indicate that none of the groups share similarity to each other. The Y-axis in plots 260, 262, and 264 is zero percent variance. If the samples were identical the distance would be close to zero. The X-axis of plots 260, 262, and 264 is a 100 percent variance. If the samples were different the distance would be close to zero.

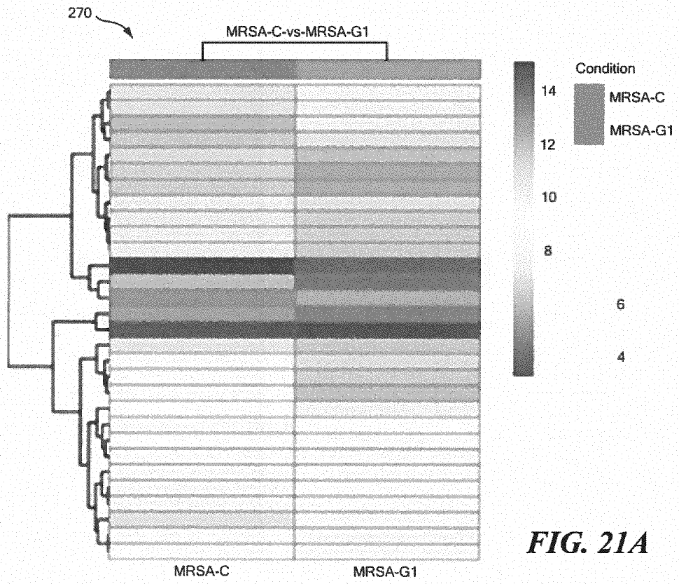

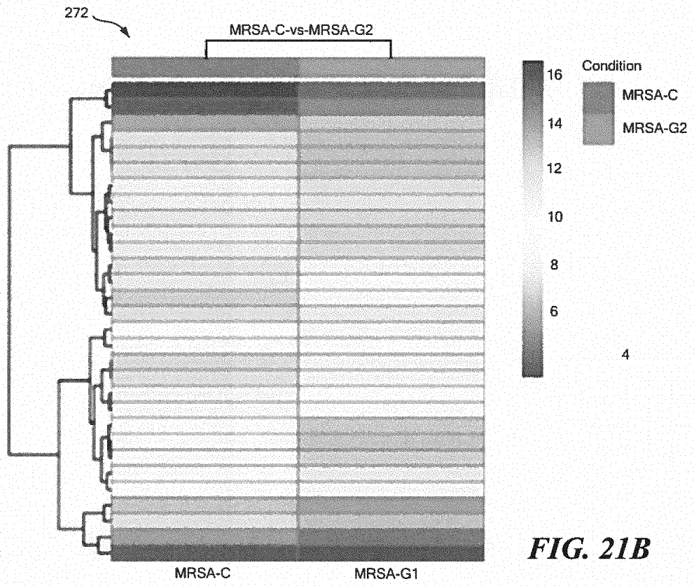

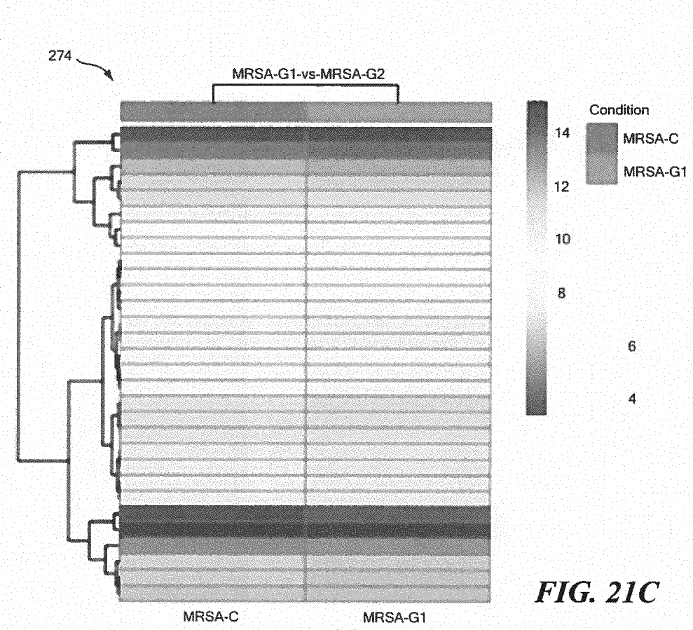

Heat map 270, FIG. 21A, shows an example of differentially expressed genes bi-clustering heat map, S. aureus ATCC 33592 Parental Wild-Type (MRSA-C) to Depigmented Generation 1 avirulent phenotype survivors (MRSA-G1) susceptible to microbicides. Heat map 272, FIG. 21B, shows an example of differentially expressed genes bi-clustering heat map, S. aureus ATCC 33592 Parental Wild-Type (MRSA-C) to Depigmented Generation 2 avindent phenotype survivors (MRSA-G2) susceptible to microbicides. Heat map 274, FIG. 21C, shows an example of differentially expressed genes bi-clustering heat map Depigmented Generation 1 avirulent phenotype survivors (MRSA-G1) susceptible to microbicides to Depigrnented Generation 2 avirulent phenotype survivors (MRSA-G2) susceptible to microbicides, normalized.

The above analysis was performed to visualize the expression profile of the top 30 genes sorted by their adjusted p-values. This analysis helps identify regulated genes across the treatment conditions. The hierarchical data bi-clustering for the three phenotypes is visualized through a similarity matrix that is based on the number of differentially expressed transcripts discovered. This similarity matrix was then used as input to a hierarchical clustering algorithm using a Euclidean distance metric and average linkage. As shown by the heat maps, 270, 272 and 274, the gene transcripts with profound overexpression profiles for the three comparisons was assessed, ranging between 4-fold to greater than 16-fold, Addition analysis was performed by annotation as discussed below.

Gene Annotation Results from RNA Sequencing:

The treated survivor strain MRSA-G1 avirulent phenotype survivors weakly express the 165 ribosomal RNA (16S rRNA) polymerase subunit used to synthesize messenger RNA. The 16S subunit binds to the Shine-Dalgarno (SD) sequence facilitating recruitment of the ribosome to messenger RNA (mRNA), a key process in the initiation of translation. Additionally, the MRSA-G1 strain is deficient in several transfer RNA (tRNA) motifs. These enzyme-like nucleotides play a critical role in translation transferring monomer peptides specified by the mRNA in complex with the polymerase leading to synthesis of a growing polypeptide chain, e.g., translation. Fourteen deficient tRNAs were found, several of which are either catalytic or modification site residues in proteins such as Glutamic acid, Serine, and Methionine, Furthermore, the MRSA-G1 strain has an impairment of the 23S ribosomal subunit, a component of the large subunit (50S) of the bacterial/archean ribosome that is also critical for the synthesis of protein that assist transient binding of the tRNA to the polymerase complex. Taken together, this S. aureus mutant is demonstrably impaired critically in its ability to perform translation. In fact, it is unlikely the MRSA-G1 avirulent phenotype survivors could effectively multiply and grow under clinically relevant conditions. These data support the inventor's observation that even under the favorable laboratory growth conditions the MRSA-G1 strain showed a significantly longer lag phase and measurable “dip” during exponential growth along with a lower density in stationary phase relative to the wild-type MRSA parental isoform. Equally important, also found was a deficiency in a hypothetical prokaryotic membrane lipoprotein lipid associated with the Type VII protein secretion (T7SS) system in S. aureus on the ess loci. The T7SS, which is a large ATP-dependent transmembrane transport system, was first identified in Mycobacterium tuberculosis with homologs having also been identified in Bacillus subtilis, Bacillus anthracis and S.au aureus. Deletion of the loci has been linked to reduced virulence in the Bacillus Calmette-Guérin (BCG) vaccine strain Mycobacterium bovis (See e.g., Hsu et al., The Primary Mechanism of Attenuation of Bacillus Calmette-Guerin is a Loss Of Secreted Lytic Function Required for Invasion of Lung Interstitial Tissue, Proc. Natl. Acad. Sci., 100(21):12420-12425 (2003), incorporated by reference herein) and evidence in murine models has demonstrated the importance of the ESX-1 system in enabling bacterial translocation from the phagolysosome into the cytosol, a key step iia mycobacterial virulence. In the commensal pathogen S. aureus, which causes the majority of skin and soft tissue infections as well as a substantial proportion of invasive infections such as endocarditis and osteomyelitis (See e.g., Lowy, F. D, Staphylococcus aureus Infections, N. Engl, Med., 339(8):520-532 (2003) and Klevens et al, Invasive Methicillin-resistant Staphylococcus aureus Infections in the United States, JAMA, 298(15):17634771 (2007), both incorporated by reference herein) the genes encoding the T7SS found on the ess loci are highly up-regulated during persistent cystic fibrosis (See e.g. Windmüller et al., Transcriptional Adaptations During Long-Term Persistence of Staphylococcus Aureus ira the AirwayS of a Cystic Fibrosis Patient, into J. Med, Microbiol, 305(1)3846 (2015), incorporated by reference herein). As a result of this survey and the observed to data, the light dose regiment of cycling steps a) and b) by system 10 and the method thereof discussed above provides an additional value beyond the scope of catheter sterilization for inactivation of biological warfare agents targeting the host respiratory system.

In one design, the light dose regiment of cycling steps a) and b) the selected number of times of system 10 and the method thereof discussed above may be performed on the skin or soft tissue of a human subject or an animal. In other designs, the light dose regiment of cycling steps a) and b) the selected number of times by system 10 and the method thereof to generate avinilent phenotype survivors susceptible to microbicides may be performed on an inert surface and is combined with the microbicides to eradicate a majority of the disease-causing germs on the inert surface.

In one example, the light dose regiment of cycling steps a) and b) the selected number of times to generate avirulent phenotype survivors susceptible to microbicides may be used to rescue failed microbicides and/or improves the effectiveness of microbicides.

The unique combination of pulsed UVA light 14, FIG. 1, and continuous UVC light 20 of step b) of system 10 and the method thereof provides a synergetic effect that results in the application of pulsed UVA light 14 and UVC light 20 at lower light energy levels when compared to applying pulsed UVA light 12 and UVC light 20 separately.

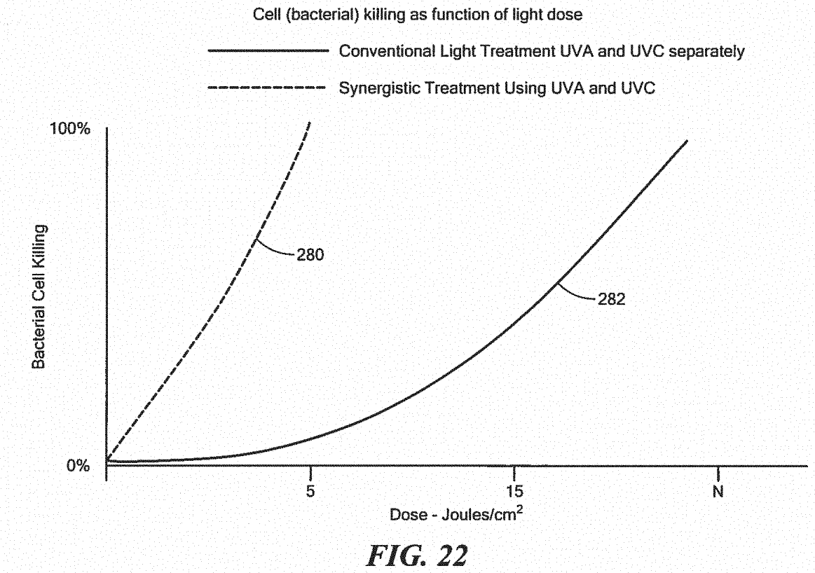

Graph 280 FIG. 22, shows an example of the lower light energy of UVA light 14 and UVC light 20 needed by system 10 and the method thereof to eradicate a majority of disease-causing germs and produce avirulent phenotype survivors susceptible to microbicides due to the synergistic effect of UVA light 14, FIG. 1, and UVC light 20. Graph 282 shows an example of the light energy needed to eradicate a majority of disease-causing germs by system 10 and the method thereof when pulsed UVA light 12 and continuous UVC light are applied separately. This feature may reduce the costs of the components of system 10.

The result is system 10 and the method thereof discussed above with reference to one or more of FIGS. 1-22 effectively and efficiently produces avirulent phenotype survivors which are susceptible to germicidal agents to overcome the problems associated with disease-causing germs becoming resistant to microhicides (discussed above). Thus, system 10 and the method produces avirulent phenotype survivors susceptible to microbial agents which can be used to provide effective medical treatment, reduce the time spent in hospitals, decrease medical costs, and reduce mortality. System 10 and the method thereof should be applicable, and render avirulent food born, environmental or livestock related pathogens. System 10 and the method thereof also may produce avirulent phenotype survivors susceptible to microbicides which are unable to produce a microbial biofilm associated with reoccurring infections, converts persisters in a microbial biofilm associated with reoccurring infections from a dormant state to a non-dormant state thereby making the persisters susceptible to microbicides, and prevents re-infection. System 10 and the method thereof interferes with gene regulation, transcription, and translation yielding an avirulent survivors phenotype that is characterized by a permanently and genetically altered state unable to maintain normal physiological gene regulation, including production of virulence factors, and germicidal resistance mechanisms to microbicides thereby rendering the avirulent phenotype survivors susceptibility to microbicides and non-infectious. System 10 and the method may be, but is not limited, used to treat Impetigo, Cellulitis, Folliculitis, Erysipelas, Necrotizing Fasciitis (Fournier's gangrene), Acute Paronychia, Erythrasma, Intertrigo, Atopic Dermatitis (eczema), Lupus vulgaris, Scrofuloderma, Tuberculosis verruca cutis (Warty tuberculosis), Tuberculids, Non-tuberculous mycobacterial infections (Endocarditis, osteomyelitis, meningitis, keratitis, Mediastinitis, catheter-related infections, regional lymphadenitis), Periodontitis, Chloranychia Dermatophytosis associated Tinea pedis (athlete's foot), Dermatophytosis associated Tinen cruris & tinea eorporis (jock itch), Dermatophytosis associated Tinen capitis, (scalp), Dermatophytosis associated Tinea corporis (body), faciei (face), and manuum (hands), Dermatophytosis associated Tinen unguium (onychomycosis), general treatment of Tinen (Pityriasis) Versicolor, Chronic paronychia, Vulvovaginal candidiasis, Candidal intertrigo, Candidal balanitis, Oral Thrush (oropharyngeal candidiasis), Complicated interdigital intertrigo, and infections associated with Intertrigo (diaper dermatitis), Atopic Eczema, Job syndrome and skin wounds

Although specific features of the invention are shown in some drawings and not in others, this is for convenience only as each feature may be combined with any or all of the other features in accordance with the invention, The words “including”, “comprising”, “having”, and “with” as used herein are to be interpreted broadly and comprehensively and are not limited to any physical interconnection. Moreover, any embodiments disclosed in the subject application are not to be taken as the only possible embodiments. Other embodiments will occur to those skilled in the art and are within the following claims.

In addition, any amendment presented during the prosecution of the patent application for this patent is not a disclaimer of any claim element presented in the application as filed: those skilled in the art cannot reasonably be expected to draft a claim that would literally encompass all possible equivalents, many equivalents will be unforeseeable at the time of the amendment and are beyond a fair interpretation of what is to be surrendered (if anything), the rationale underlying the amendment may bear no more than a tangential relation to many equivalents, and/or there are many other reasons the applicant cannot be expected to describe certain insubstantial substitutes for any claim element amended.

What is claimed is:

1. A system to eradicate a majority of disease-causing germs and produce avirulent phenotype survivors susceptible to microbicides using a light dose regiment, the system comprising:
one or more processors;
one or more light sources coupled to the one or more processors, the one or more processors configured to control the one or more light sources to apply the light dose regiment including:
a) continuously applying a pre-treatment of non-lethal UVA light to the disease causing germs to increase the production of pigment to the disease-causing germs;
b) simultaneously applying pulsed UVA light and continuous UVC light to the disease-causing germs having the increased pigment therein to eradicate the majority of the disease-causing germs; and
c) cycling steps a) and b) a selected number of times to generate avirulent phenotype survivors susceptible to microbicides.

2. The system of claim 1 in which cycling steps a) and b) the selected number of times to generate the avirulent phenotype survivors susceptible to microbicides is based on the number of disease-causing germs and the distance the disease causing germs are from the one or more light sources.

3. The system of claim 1 in which the light dose regiment includes a number of times steps a) and b) are performed.

4. The system of claim 1 in which characteristics of the light dose regiment includes one or more of: a wavelength of the UVA light and/or a wavelength of the UVC light, a wavelength of waveform of the UVA light and/or a waveform of the UVC light, a light power of the UVA light and/or a light power UVC light and/or an amount of time each of steps a) and b) are performed.

5. The system of claim 1 in which the germs include one or more of disease-causing bacteria and fungi.

6. The system of claim 1 in which the microbicides include one or more of: antibiotics, aritifungal agents, and antiseptics.

7. The system of claim 1 in which avirulent phenotype survivors susceptible to microbicides include disease-causing bacteria and fungi.

8. The system of claim in which cycling steps a) and b) the selected number of times produces the avirulent phenotype survivors susceptible to microbicides which are stable over multiple generations.

9. The system of claim 1 in which cycling steps a) and b) the selected number of times produces the avirtilent phenotype survivors susceptible to microbicides which are unable to produce a microbial biofilin associated with reoccurring infections.

10. The system of claim (in which cycling steps a) and b) the selected number of times converts persisters in a microbial biotilm associated with reoccurring infections from a dormant state to a non-dormant state thereby making the persisters susceptible to microbicides.

11. The system of claim i in which cycling steps a) and b) the selected number of times interferes with gene regulation, transcription, and translation yielding an avirulent survivors phenotype that is characterized by a permanently and genetically altered state unable to maintain normal physiological gene regulation, including production of virulence factors, and germicidal resistance mechanisms to mircrobicides thereby rendering the avirulent phenotype survivors susceptibility to microbicides and non-infectious.

12. The system of claim 1 in which cycling steps a) and b) the selected number of times is performed on the skin or soft tissue of a human subject or an animal.

13. The system of claim 1 in which the cycling steps a) and b) the selected number of times is performed on an inert surface and is combined with the microbicides to eradicate a majority of the disease-causing germs on the inert surface.

14. The system of claim 1 in which the avirulent germicide susceptible phenotype survivors are stable over multiple generations.

15. The system of claim I in which cycling steps a) and b) the selected number of times to generate aviruient phenotype survivors susceptible to microbicides rescues failed microbicides and/or improves the effectiveness of microbicides.

16. A method to eradicate a majority of disease-causing germs and produce avirulent phenotype survivors susceptible to microbicides with a light dose regiment, the method comprising:
a) continuously applying a pre-treatment of non-lethal UVA light to the disease-causing germs to increase the production of pigment to the disease-causing germs;
b) simultaneously applying pulsed UVA light and continuous UVC light to the disease-causing germs having the increased pigment therein to eradicate the majority of the disease-causing germs; and
c) cycling steps a) and b) a selected number of times to generate avirulent phenotype survivors susceptible to microbicides.

17. The method of claim 16 in which cycling steps a) and b) the selected number of times to generate the avirulent phenotype survivors susceptible to microbicides is based on the number of disease-causing germs and the distance the disease-causing germs are from the one or more light sources.

18. The method of claim 16 in which the light dose regiment includes a predetermined number of times steps a) and b) are performed.

19. The method of claim 16 in which characteristics of the light dose regiment includes one or more of: a wavelength of the UVA light and/or a wavelength of the UVC light, a waveform of the UVA light and/or a waveform of the UVC light, a light power of the UVA light and/or a light power of the UVC light and/or an amount of time each of steps a) and b) are performed.

20. The method of claim 16 in which the getins include one or more of disease-causing bacteria and fungi.

21. The method of claim 16 in which the microbicides include one or more of: antibiotics, antifungal agents, and antiseptics.

22. The method of claim 16 in which avirulent phenotype survivors susceptible to microbicides include disease-causing bacteria and fungi.

23. The method of claim 16 in which cycling steps a) and b) the selected number of times produces the avirulent phenotype survivors susceptible to microbicides which are stable over multiple generations.

24. The method of claim 16 in which cycling steps a) and b) the selected number of times produces the avirulent phenotype survivors susceptible to microbicides which are unable to produce a microbial biofilms associated with reoccurring infections.

25. The method of claim 16 in which cycling steps a) and b) the selected number of times converts persisters in a microbial biofilm associated with reoccurring infections from a dormant state to a non-dormant state thereby making the persisters susceptible to microbicides.

26. The method of claim 16 in which cycling steps a) and b) the selected number of times interferes with gene regulation, transcription, and translation yielding a avirulent phenotype survivors phenotype which permanently and genetically alters the virulent phenotype survivors phenotype to produce the avirulent phenotype survivors susceptible to mircrobicides and renders the avirulent phenotype survivors unable to maintain normal physiological function including the regulation and production of virulence factors and increases the avirulent phenotype survivors susceptibility to microbicides.

27. The method of claim 16 in which cycling steps a) and b) the selected number of times is performed on the skin or soft tissue of a human subject or an animal.

28. The method of claim 16 in which the cycling steps a) and b) the selected number of times is performed on an inert surface and is combined with the microbicides to eradicate a majority of the disease-causing germs on the inert surface.

29. The method of claim 16 in which the avirulent germicide susceptible phenotype survivors are stable over multiple generations.

30. The method of claim 16 in which cycling steps a) and b) the selected number of times to generate avirulent phenotype survivors susceptible to microbicides rescues failed microbicides and/or improves the effectiveness of microbicides.
